(19) United States
(12) Patent Application Publication
(10) Pub. No.: US 2022/0230217 A1
(43) Pub. Date: Jul. 21, 2022

(54) SENIOR LIVING ENGAGEMENT AND CARE SUPPORT PLATFORMS WITH GROCERY DELIVERY

(71) Applicant: State Farm Mutual Automobile Insurance Company, Bloomington, IL (US)

(72) Inventors: Victoria Wallace, Brookhaven, GA (US); Daniel Wang, Las Vegas, NV (US); Nikhil Patel, San Jose, CA (US); Arjun Saxena, Sunnyvale, CA (US); Diana Sherwood, Scottsdale, AZ (US); Marcus E. Rohlfs, San Bruno, CA (US); Emily R. Bryant, Mesa, AZ (US)

(21) Appl. No.: 17/578,207

(22) Filed: Jan. 18, 2022

Related U.S. Application Data
(60) Provisional application No. 63/186,599, filed on May 10, 2021.
(60) Provisional application No. 63/161,274, filed on Mar. 15, 2021.
(60) Provisional application No. 63/139,107, filed on Jan. 19, 2021.

(51) Int. Cl. G06Q 30/06; G06Q 10/08; G16H 20/60

(52) U.S. Cl. CPC G06Q 30/0613; G06Q 30/0633; G16H 20/60; G06Q 10/0834; G06Q 10/087; G06Q 30/0631

(57) ABSTRACT

Provided herein is a ECSP computer device for facilitating senior user engagement. The processor is programmed to: (i) store user registration information for a senior user associated with the first client device; (ii) store caregiver registration information for a plurality of caregivers associated with the senior user, wherein the caregiver registration information includes data for identifying the at least one second client device; (iii) receive, from at least one of the senior user and the plurality of caregivers, one or more items of an order of the senior user; (iv) compile the received items into a list; (v) generate the order based on user preferences and the list; (vi) determine at least one third party to fulfill the order based on the items on the list and the user preferences; and (vii) transmit the order to a third party server associated with the determined third party.

### CROSS-REFERENCE TO RELATED APPLICATIONS

This patent application claims priority to U.S. Provisional Patent Application 63/186,599, May 10, 2021, entitled “SENIOR LIVING ENGAGEMENT AND CARE SUPPORT PLATFORMS WITH GROCERY DELIVERY,” to U.S. Provisional Patent Application Ser. No. 63/161,274, filed Mar. 15, 2021, entitled “SENIOR LIVING ENGAGEMENT AND CARE SUPPORT PLATFORMS WITH GROCERY DELIVERY,” and to U.S. Provisional Patent Application Ser. No. 63/139,107, filed Jan. 19, 2021, entitled “SENIOR LIVING ENGAGEMENT AND CARE SUPPORT PLATFORMS WITH GROCERY DELIVERY,” and is related to U.S. patent application Ser. No. 16/996,592, filed Aug. 18, 2020, entitled “SENIOR LIVING ENGAGEMENT AND CARE SUPPORT PLATFORMS,” which claims the benefit of priority to U.S. Provisional Patent Application Ser. No. 63/041,409, filed Jun. 19, 2020, entitled “SENIOR LIVING ENGAGEMENT AND CARE SUPPORT PLATFORMS,” U.S. Provisional Patent Application Ser. No. 62/935,854, filed Nov. 15, 2019, entitled “SENIOR LIVING ENGAGEMENT AND CARE SUPPORT PLATFORMS,” U.S. Provisional Patent Application Ser. No. 62/935,860, filed Nov. 15, 2019, entitled “SENIOR LIVING ENGAGEMENT AND CARE SUPPORT PLATFORMS,” U.S. Provisional Patent Application Ser. No. 62/888,746, filed Aug. 19, 2019, entitled “SENIOR LIVING ENGAGEMENT AND CARE SUPPORT PLATFORMS,” and U.S. Provisional Patent Application Ser. No. 62/892,207, filed Aug. 27, 2019, entitled “SENIOR LIVING ENGAGEMENT AND CARE SUPPORT PLATFORMS,” the entire contents and disclosures of which are hereby incorporated herein by reference in their entirety.

### FIELD OF THE DISCLOSURE

The present disclosure relates to senior living computer platforms and, more particularly, to systems and methods for using a senior living computer platform to facilitate senior engagement with their daily schedule and caregivers associated with the seniors, and coordinate care between caregivers, while providing a grocery delivery platform.

### BACKGROUND

At least some conventional computer networks have enabled caregivers (e.g., family members, friends, and care service providers) associated with senior users to coordinate care for the senior user. However, conventional systems usually merely keep a schedule of the coordinated care, and may not provide additional functionality. Further, known systems may not facilitate senior engagement in their daily schedules, and may therefore not provide information on such engagement to caregivers. Known systems may have other drawbacks as well.

### BRIEF SUMMARY

The present embodiments may relate to systems and methods for facilitating senior engagement in their daily schedules and coordinating care between caregivers of the senior. The system may include an engagement and care support computing device, one or more client devices, one or more third party servers, and/or one or more databases.

In one aspect, an engagement and care support platform computer system for facilitating senior user engagement may be provided. The computer system may include at least one processor in communication with at least one memory device. The at least one processor may be programmed to: (i) register a user through an application, wherein the user inputs personal and scheduling data into the application, (ii) register a caregiver associated with the user through the application, (iii) generate a senior profile based upon the user personal and scheduling data, (iv) build a daily interactive user interface that reflects the senior profile, (v) display the daily interactive user interface at a first client device associated with the user, (vi) cause the first client device to initiate a daily interaction prompt to the user, (vii) determine whether any user interaction was received at the first client device in response to the daily interaction prompt, and (viii) transmit a daily update message to a second client device associated with the caregiver, the daily update message including an indication of whether any user interaction was received at the first client device. The care coordination support platform computer system may include additional, less, or alternate functionality, including that discussed elsewhere herein.

In another aspect, a computer-implemented method for facilitating senior user engagement may be provided. The computer-implemented method may be performed by an engagement and care support platform computer system including at least one processor in communication with at least one memory device. The computer-implemented method may include: (i) registering a user through an application, wherein the user inputs personal and scheduling data into the application, (ii) registering a caregiver associated with the user through the application, (iii) generating a senior profile based upon the user personal and scheduling data, (iv) building a daily interactive user interface that reflects the senior profile, (v) displaying the daily interactive user interface at a first client device associated with the user, (vi) causing the first client device to initiate a daily interaction prompt to the user, (vii) determining whether any user interaction was received at the first client device in response to the daily interaction prompt, and/or (viii) transmitting a daily update message to a second client device associated with the caregiver, the daily update message including an indication of whether any user interaction was received at the first client device. The method may include additional, less, or alternate actions, including those discussed elsewhere herein.

In yet another aspect, a non-transitory computer-readable media having computer-executable instructions embodied thereon may be provided. When executed by an engagement and care support platform computer system including a processor in communication with a memory device, the computer-executable instructions may cause the processor to (i) register a user through an application, wherein the user inputs personal and scheduling data into the application, (ii) register a caregiver associated with the user through the application, (iii) generate a senior profile based upon the user personal and scheduling data, (iv) build a daily interactive user interface that reflects the senior profile, (v) display the daily interactive user interface at a first client device associated with the user, (vi) cause the first client device to initiate a daily interaction prompt to the user, (vii) determine whether any user interaction was received at the first client device in response to the daily interaction prompt, and (viii) transmit a daily update message to a second client device associated with the caregiver, the daily update message including an indication of whether any user interaction was received at the first client device.

In still a further aspect, a ECSP computer device for facilitating senior user engagement may be provided. The ECSP computer device may include at least one processor in communication with at least one memory device. The ECSP computer device may be in communication with a plurality of second client devices. The at least one processor may be programmed to (i) store user registration information for a user associated with the first client device; (ii) store caregiver registration information for a plurality of caregivers associated with the user, wherein the caregiver registration information includes data for identifying the plurality of second client devices; (iii) receive, from at least one of the user and the plurality of caregivers, one or more items of an order of the user; (iv) compile the received items into a list; (v) generate the order based on user preferences and the list; (vi) determine at least one third party to fulfill the order based on the items on the list and the user preferences; and/or (vii) transmit the order to a third party server associated with the determined third party. The ECSP computer device may include additional, less, or alternate functionality, including that discussed elsewhere herein.

In yet a further aspect, a computer-implemented method for facilitating senior engagement may be provided. The method may be implemented by a computer device including at least one processor and/or transceiver in communication with at least one memory device. The method may include, via the computer device and/or one or more local or remote processors and associated transceivers, (i) storing user registration information for a user associated with a first client device; (ii) storing caregiver registration information for a plurality of caregivers associated with the user, wherein the caregiver registration information includes data for identifying the plurality of second client device associated with the corresponding caregiver; (iii) receiving, from at least one of the user and the plurality of caregivers, one or more items of an order of the user; (iv) compiling the received items into a list; (v) generating the order based on user preferences and the list; (vi) determining at least one third party to fulfill the order based on the items on the list and the user preferences; and/or (vii) transmitting the order to a third party server associated with the determined third party. The method may include additional, less, or alternate actions, including those discussed elsewhere herein.

### BRIEF DESCRIPTION OF THE DRAWINGS

The Figures described below depict various aspects of the systems and methods disclosed therein. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and are instrumentalities shown, wherein:

FIG. 1 illustrates an exemplary engagement and care support computer system for facilitating engagement of a user and caregivers with a care schedule of the user;

FIG. 2 illustrates an exemplary configuration of an exemplary user computing device that may be used in the engagement and care support computer system illustrated in FIG. 1;

FIG. 3 illustrates an exemplary configuration of an exemplary server computing device that may be used in the engagement and care support computer system illustrated in FIG. 1;

FIG. 4 illustrates a flow chart of an exemplary computer-implemented method implemented by the exemplary engagement and care support computer system shown in FIG. 1;

FIG. 5 illustrates a diagram of components of one or more exemplary computing devices that may be used in the engagement and care support computer system shown in FIG. 1;

FIG. 6 is a screenshot of one example initial welcome page of an engagement and care support application illustrated in FIG. 1;

FIG. 7 is a screenshot of one example user registration page of an engagement and care support application illustrated in FIG. 1;

FIG. 8 is a screenshot of one example caregiver registration page of an engagement and care support application illustrated in FIG. 1;

FIG. 9 is a screenshot of one example welcome page of an engagement and care support application illustrated in FIG. 1;

FIG. 10 is a screenshot of one example user care team page of an engagement and care support application illustrated in FIG. 1;

FIG. 11A is a screenshot of one example user home page of an engagement and care support application illustrated in FIG. 1;

FIG. 11B is a screenshot of another example user home page of an engagement and care support application illustrated in FIG. 1;

FIGS. 11C-11L are screenshots of example user interaction pages of an engagement and care support application illustrated in FIG. 1;

FIGS. 12A and 12B are screenshots of example caregiver home screens of an engagement and care support application illustrated in FIG. 1;

FIGS. 13A and 13B are screenshots of further example caregiver home screens of an engagement and care support application illustrated in FIG. 1;

FIG. 14 is a screenshot of one example caregiver feed page of an engagement and care support application illustrated in FIG. 1;

FIG. 15 is a screenshot of one example caregiver schedule page of an engagement and care support application illustrated in FIG. 1;

FIGS. 16 and 17 are screenshots of example caregiver list pages of an engagement and care support application illustrated in FIG. 1;

FIG. 18 is a screenshot of one example care circle page of an engagement and care support application illustrated in FIG. 1;

FIG. 19 illustrates an exemplary computer-implemented method of providing a care circle platform that includes chatbot and mobile application functionality that facilitates coordination of virtual care circle member communication and tasks;

FIG. 20 depicts a view of an exemplary enhanced engagement and care support platform system that may be used to facilitate engagement of a user and coordinating care of the user between caregivers associated with the user that includes an enhancement to allow for grocery delivery to the user; and

FIGS. 21A and 21B illustrate a flow chart of an exemplary computer-implemented method for ordering and having groceries delivered implemented by the exemplary enhanced engagement and care support computer system shown in FIG. 20.

The Figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION

The present embodiments may relate to, inter alia, systems and methods for facilitating engagement of a senior user (also referred to herein as a “user”) in a care schedule of the user and coordinating care between caregivers associated with the user. In one exemplary embodiment, the process may be performed by an engagement and care support platform (“ECSP”) computer system (also referred to herein as an “ECSP platform” and an “ECSP server”). In another embodiment, the process may be performed by a digital care circle platform, which may be configured to perform steps that are substantially similar to those described herein for the ECSP computer system.

As described below, the systems and methods described herein may leverage different types of data (e.g., user and caregiver data, user events including tasks, activities, and appointments, caregiver schedules, smart device data, and mobile device data) to facilitate independent engagement of a user (e.g., a senior user) and provide information associated with that information to a caregiver.

The caregivers associated with the user may include people who normally take care of the user (e.g., family members, friends, paid caregivers, etc.) and service providers of the user (e.g., health care professionals, such as doctors, nurses, physical therapists, occupational therapists, etc.). The caregivers often have busy schedules, and it may be difficult for the caregivers to coordinate caring for the user. Accordingly, friction between caregivers may arise from constantly trying to coordinate care and scheduling to take care of the user. Moreover, it can be unclear whether or when certain tasks have been completed, which may lead to redundancy in task completion and/or incomplete tasks.

In addition, some senior users may desire a level of independence from their caregivers, and may only want or need assistance for certain tasks. Accordingly, the user may grow frustrated with unpredictable caregiver schedules and/or unnecessary caregiver presence.

To assist the senior users in having more independence, enhanced engagement and care support platform (EECSP) computer systems (also referred to herein as an “EECSP platform” and an “EECSP server”) can also interface with grocery delivery services. The EECSP computer system may assist the senior in building a grocery/shopping list. Then the EECSP computer system may assist the senior user by placing an order with one or more grocery delivery services to have the groceries on the list be delivered to the senior user. The EECSP computer system may also allow members of the senior's circle of care to add items to the grocery/shopping list. The members of the senior's circle of care may also be able to initiate purchase of the groceries and even pay for the groceries. While the above describes adding groceries to a grocery/shopping list, the user may keep track of multiple shopping lists, such as a holiday shopping list or a home maintenance shopping list. Ones having skill in the art would understand that many of the steps for ordering groceries may also be used for ordering other, non-grocery items.

The EECSP computer system may also monitor the grocery purchases of the senior users to determine when certain items need to be reordered. The EECSP computer system may automatically place items on the grocery/shopping list after a specific period of time has passed since the items were last purchased. These specific periods of time may be based on consumption rates from the individual senior, or based on seniors overall. The EECSP computer device may also track expiration dates, to reorder foods that are about to expire or have already expired. The EECSP computer system may also be connected to sensors, such as a smart refrigerator, to determine which foods the senior currently has and may use that information to determine which items to add to the grocery/shopping list and when to order those items.

The systems and methods described herein ensure that the user is actively engaged in their care schedule and that each caregiver is informed about the status of the user and their assigned tasks of the care schedule. In addition, caregivers are able to care for the user and/or carry out tasks for the user and may reduce friction between caregivers by providing a platform that automatically assigns care duties to caregivers based upon information (e.g., scheduling and calendar information) input by the caregivers and ensures that the caregivers complete their assigned duties. Further, the systems and methods described herein may learn about the user and associated caregivers and adjust interactions with the user and associated caregivers and the coordinating of the care schedule of the user as well as engagement of the user based upon the learning. Moreover, the systems and methods herein facilitate independent senior engagement with an interface that enables caregivers to remotely view the user's interactions with the interface. Therefore, the caregivers can be assured of the senior user's state.

### Exemplary User and Caregiver Data Collection

In the exemplary embodiment, an engagement and care support platform (e.g., provided by an engagement and care support platform server) may leverage different kinds of data (e.g., user and caregiver data, user events, caregiver schedules, sensor data, and mobile device data) to coordinate a care schedule of a user between one or more caregivers associated with the user and/or promote user engagement with the engagement and care support platform. In the exemplary embodiment, a primary caregiver (e.g., an admin caregiver) may register for the engagement and care support platform (“ECSP”) service provided by an ECSP server through an application (e.g., a ECSP application) on a mobile device associated with the admin caregiver, or any other suitable device that may access the ECSP application and/or a website associated with the ECSP application. The user may also register for the ECSP application for himself or herself.

In registering for the ECSP service, the admin caregiver may provide the ECSP server with information associated with the user. The information associated with the user may include user data (e.g., name, birthdate, height, weight, etc.), user tasks (e.g., taking medicine, bathing, eating, paying bills, getting groceries, car maintenance, home maintenance, etc.), user activities (e.g., social activities, like bingo and golfing, physical activities, like working out and keeping active, etc.), user interests and hobbies (e.g., fishing, home improvement, gardening, etc.) user appointments (e.g., recurring appointments like yearly physicals and bimonthly haircuts, etc.), user alert preferences (e.g., when and through which method users prefer to be alerted), and any other information associated with the user that may be useful to the ECSP server. This information associated with the user may be stored in a “senior profile,” which may be leveraged to generate activity schedules, provide relevant content (e.g., articles or games), and the like. In other embodiments, the user may register for the service and provide the ECSP server with information for the senior profile themselves. In some embodiments, the admin caregiver and/or the user may provide contact information for users that may not be caregivers, such as non-caregiver family members, social groups (e.g., member of a book club), etc. The contact information for these users may be identified and stored using “shortcut” names or phrases that identify the group. For example, contact information for a user's three children may be identified collectively as a “Kids” group.

Further, in registering for the ECSP service, the admin caregiver may invite other caregivers to be a part of a care team for the user and/or the user may invite other caregivers himself or herself. For example, the admin caregiver may provide the ECSP server with a list of emails and/or phone numbers of other caregivers who are associated with the user. The ECSP server may send an invitation link and/or code to the other caregivers instructing the other caregivers on how to sign up for the care team for the user associated with the admin caregiver.

Each caregiver, including the admin caregiver, may register themselves for the ECSP service. In registering for the ECSP service, the caregivers may provide the ECSP server with caregiver information (e.g., name, contact information, relationship to the user), caregiver schedule information (e.g., known work and/or activity schedules of the caregivers), caregiver alert preferences (e.g., when and through which method caregivers prefer to be alerted), and/or any other caregiver information that may be useful to the ECSP server. In some embodiments, the caregivers may link their digital calendars (e.g., provided on a mobile device associated with the caregiver) to the ECSP server such that the caregivers do not have to manually input scheduling data available to the ECSP server into the digital calendar. The user and the caregivers may update and/or edit the user and caregiver data at any time (e.g., through the ECSP application). Further, the admin caregiver and/or the user may require that each registered caregiver be approved before the caregivers are officially added to the caregivers of the user by the ECSP application. For example, the ECSP application may push a notification to the mobile device associated with the admin caregiver and/or a user device associated with the user each time a new caregiver is registered. The notification may prompt the admin caregiver and/or the user to accept or deny (e.g., through a push notification or voice command) the new caregiver. If the new caregiver is accepted by the user and/or the admin caregiver, the new caregiver may be automatically added to the caregivers associated with the user by the ECSP application. As described herein, notifications may include, but are not limited to, SMS (Short Message Service), MMS (Multimedia Messaging Service), text message, chat message, instance message, email, push notification, in-app message, rich notifications, and rich push notifications.

In the exemplary embodiment, if the caregiver is a person who normally takes care of the user and needs to view and/or be notified of the schedule of the user, the caregiver may fully register for the ECSP service. If the caregiver is a person who only provides certain services to the user (e.g., a doctor, nurse, physical therapist, occupational therapist, etc.) and/or is socially involved with the user for specific activities (e.g., a garden club member, a book club member, etc.), the caregiver may have very limited access to the ECSP service, and the ECSP server may have very limited access to the caregiver data (e.g., the ECSP server may simply receive calendar updates from the caregiver if an event related to the user is scheduled).

In the exemplary embodiment, the ECSP server may also be configured to receive sensor data from sensors associated with the user and/or the caregivers. For example, sensors may include smart home device sensors (e.g., AMAZON ALEXA, GOOGLE HOME, and/or RING doorbells), wearable device sensors (e.g., APPLE WATCH and FITBIT), smart device sensors (e.g., smart pillboxes), sensors associated with a mobile device of the user and caregivers (e.g., GPS sensors), and any other sensors. In the exemplary embodiment, the ECSP server may be configured to store the received data (e.g., user data, caregiver data, sensor data, etc.) in a memory.

In some embodiments, the EECSP computer system or the ECSP computer system is in communication with one or more sensors that inform the computer system about the current state of the foods that the senior has. For example, the sensor may be a smart refrigerator, and can tell the system how many and which foods the senior has in the smart refrigerator. The sensors may also include one or more Internet of Things (IoT) devices that monitor food storage, such as, but not limited to an IoT egg tray that can count the number of eggs remaining, IoT food storage containers that can alert users when the contents of their refrigerator are about to spoil, or an IoT pantry that tracks current inventory.

### Exemplary Care Coordination

In the exemplary embodiment, the ECSP server may be configured to process all of the user and caregiver data (e.g., events of the user, and schedules and preferences of the caregivers) the ECSP server receives from the user and caregivers (e.g., through the ECSP application) and coordinate a care schedule of the user between the caregivers and/or promote user engagement with the ECSP application. The ECSP server may automatically assign each task, activity, and/or appointment of the user to the caregivers based upon the received caregiver data, and the received caregiver data may include caregiver preferences and schedules. The ECSP server may also promote user engagement with the ECSP server by “learning” about the user from the received user data and suggest different types of media (e.g., articles, videos, movies, TV shows, etc.) that may interest the user. The ECSP server may further suggest different activities based on the “learning” about the user. For example, the ECSP server may suggest and/or send a link to an online invite for a new gardening club if the user typically reads about gardening. The EECSP server or ECSP server may also determine which foods that the senior user likes based on past ordering. The EECSP server may then suggest recipes or sales for the determined foods.

The EECSP server or ECSP server may also determine standard food orders for the user (i.e., the senior user) to enhance their grocery and food ordering experience. In at least one embodiment, the user adds items to their grocery shopping list over the week through the EECSP server or ECSP server. The user may have preset a preferred service and/or store for delivery. The user may then tell the EECSP server or the ECSP server to order groceries. The EECSP server or the ECSP server may use the grocery/shopping list to generate a grocery/shopping order and mark the ordered items as either ordered or complete. In some embodiments, the EECSP server or the ECSP server waits until the items are delivered until they are marked complete. In some embodiments, the complete items are removed from the user's view of the list. Or the items are marked with a strikethrough, check mark, or other acknowledgement that they have been ordered. In some embodiments, the ordered items on the grocery/shopping list are acknowledged with a special marking and remain on the list for a period of time, such as three days. If one or more items may not be ordered, such as those items that are out of stock, those items are left on the list. The EECSP server or the ECSP server may ask the user to confirm ordering groceries if the list is short, such as three or fewer items. After generating the grocery/shopping order, the EECSP server or the ECSP server asks the user to confirm the order. When the user confirms the order, the EECSP server or the ECSP server places the grocery/shopping order. In a later embodiment, the EECSP server or the ECSP server may store a standard order for the user and autopopulate the user's grocery/shopping order with the items from the standard order. The standard order may be generated by the EECSP server or the ECSP server based on the historical grocery purchases by the user. The EECSP server or the ECSP server may also track the consumption of food by the user, and may then remind the user to place a grocery/shopping order when food supplies are getting low. The EECSP server or the ECSP server may also determine a day(s) of the week or the month that the user normally orders groceries and prompts the user asking if they want to order groceries on that day.

The ECSP server may allow caregivers to easily assign tasks, activities, and events of the user amongst themselves such that each caregiver is informed about the care schedule of the user, including which caregiver is assigned to each task of the care schedule. For example, the primary caregiver of the user may assign tasks that the primary caregiver knows each caregiver can handle and/or the caregivers can assign tasks to themselves as the tasks are created (e.g., through the ECSP application). The ECSP server may also process the caregiver data associated with the caregivers and compare the caregiver data to the task, activity, and appointment schedule of the user. Based upon the compared data, the ECSP server may assign each task, activity, and appointment of the user to each of the caregivers. For example, the ECSP server may assign the events to the primary caregiver first (based upon a schedule of the primary caregiver) and then assign the rest of the events not assigned to the primary caregiver to other caregivers based upon the schedules of the other caregivers.

Further, the user and/or caregivers may assign some events to the user to carry out when the user does not need assistance with the events (e.g., taking medication, doing nightly security checks, and/or doing daily exercises). Once the events have been assigned to the user and/or the caregivers, the ECSP server may create a care schedule of the user. The care schedule may include all of the user's events, and the user and/or caregiver assigned to the events. The care schedule may be stored in, for example, a care database, in a memory device associated with the ECSP server.

The ECSP server may determine if events of the care schedule of the user are taken care of by the assigned caregiver through, for example, sensor data received by the ECSP server. For instance, if a user is scheduled to take medicine at a certain time two times a day, the ECSP server may receive data from a sensor (e.g., a smart pillbox) associated with the user to determine if the pill box was opened at the certain scheduled times. Further, for example, if a caregiver is scheduled to take the user to a doctor's appointment at a certain time, the ECSP server may receive location data of the user and the caregiver (e.g., from mobile devices of the user and/or caregiver) to determine if the caregiver took the user to the doctor's appointment. Additionally, if the user is scheduled to receive a grocery delivery at a certain time, the ECSP server may receive data (e.g., from a smart home device like a smart doorbell) to determine if the groceries were delivered for the user (e.g., through determining if the doorbell was rung and/or a delivery person showed up around the scheduled time).

If the ECSP server determines that a task, activity, and/or appointment has not been carried out, the ECSP server may alert (e.g., through the ECSP application) the user and/or caregivers based upon the user and caregiver data (e.g., alert preferences). Further, the ECSP server may notify the caregivers, based on the alert preferences of the caregivers, when a scheduled event has been carried out by the caregiver and/or others (e.g., service providers). In other embodiments, the user and caregivers may manually enter that the events of the user have been taken care of by the caregiver.

The EECSP server or ECSP server may also send notifications out to the members of the circle of care when a grocery/shopping order is placed and/or delivered to the senior user. Notifications may also be sent to the senior user when a circle of care member adds an item to the grocery/shopping list. The notification may include an option for the senior to approve putting the item on the grocery/shopping list. Notifications may be sent to members of the senior's circle of care when items are added to the grocery/shopping list, when items are removed from the grocery/shopping list, when an order is placed, and/or when the order is received.

### Exemplary Care Coordination Support Application

In the exemplary embodiment, an ECSP application is associated with the ECSP server. The ECSP application may be configured to receive user and caregiver data, display the care schedule of the user to the caregivers, and/or alert and/or notify the user and caregivers of assigned events. The ECSP application may be run on a device associated with the user and/or caregiver (e.g., a mobile device and/or laptop of the user or caregiver). The ECSP application may be configured to display the care schedule of the user based upon the preference of the user and caregivers. For example, the ECSP application may display a list of daily, weekly, and/or monthly tasks assigned to the user and/or caregivers, a calendar that marks when the user and/or caregiver has assigned events, and any other display method that allows the user and caregivers to easily see and interact with the care schedule of the user.

In the exemplary embodiment, the ECSP server may include a chatbot that is embedded in the ECSP application and has access to the information stored by the ECSP server (e.g., scheduled/assigned events, user data, caregiver data, etc.). The chatbot may be any suitable chatbot and/or robo-assist device that functions as described herein. The chatbot may assist the user in interacting with the ECSP application (e.g., the chatbot may recognize voice commands and/or typed commands from the user), the user and caregivers in adding, editing, and/or deleting user and caregiver data, coordinating care of the user between the caregivers, receiving information about the assigned care schedule, and/or receiving information about how the user and caregivers are carrying out the care schedule. For example, instead of a user having to physically check-in with the ECSP application (e.g., through a user interface of the device associated with the user) and/or the user and/or caregiver having to manually input each event of the user, each notification request of the user and caregivers, and/or each schedule item of the caregivers, the user and caregivers may give instructions to the chatbot (e.g., through typing and/or speaking commands and/or questions using plain or colloquial language, rather than structured commands, into the chatbot through the ECSP application). For example, the chatbot may interpret the user and/or caregivers saying, “Go home,” “Go to my dashboard,” “Go to my home screen,” or “Return home” all as commands to navigate to a dashboard of the ECSP application. Additionally, “Show me my care circle,” “View my care circle,” “Who's in my care circle,” and “Show people in my care circle” may be interpreted by the chatbot as commands to view the care circle, “Help,” “Help me,” “Show me the customer support number,” and “Contact customer support” may be interpreted by the chatbot as commands to get help from customer service associated with the ECSP application, “Approve,” “I want to approve [name of caregiver],” “Allow,” and “Allow [name of caregiver] to join” may be interpreted by the chatbot as commands to approve new caregivers, etc. Accordingly, the chatbot may be able to commands in the form of plain and colloquial language from the user and caregivers into actions. Also, for example, if the user just added a daily medication to their routine, the user may instruct the chatbot to add the medication to the daily list of tasks for the user to carry out. Further, a caregiver may instruct the chatbot that the user's lawn needs to be mowed every week in the summer.

The chatbot may be an application executed by the ECSP application or may be a separate (potentially third-party) application in communication with the ECSP application, such as through a ECSP server. The chatbot is programmed to manage communications between the ECSP application and the user. The user speaks an audible word or phrase which is collected by a microphone or other audio input device associated with the ECSP computer system and/or a client system associated with the user. The chatbot receives the audio signals including the audible word or phrase. The chatbot parses the audible word or phrase from the audio signals. In some embodiments, the chatbot is programmed to filter out additional audible sounds, such as background noise and animals, to detect human speech. In other embodiments, the chatbot is further programmed to detect specific noises, such as a person falling or breaking glass or other objects to alert the ECSP application that the user may be in trouble. The chatbot is programmed to separate these noises from human speech to recognize when the user is communicating with the ECSP application.

The chatbot transforms the audible words into text to determine what the user said. In some embodiments, the chatbot generates a response. In other embodiments, the chatbot accesses the ECSP application and/or an ECSP server to retrieve information to include in the response. For example, if the user asks what activities they have scheduled for the day, the chatbot transmits a request to the ECSP application and/or an ECSP server to retrieve the user's schedule from the calendar component. The chatbot then uses that information to generate replies to the user, where the replies are formatted in plain language. For example, the chatbot may generate a reply of “You have a tennis match at 3 pm today at the gym.” This reply is routed to the ECSP application and played through one or more speakers or other audio outputs of the ECSP computer system. If the user has more than one ECSP computer system associated with the ECSP application, the chatbot and/or the ECSP server routes the response to the ECSP computer system that received the query. In the exemplary embodiment, the chatbot is programmed to receive and respond with natural language processing, so that the user does not have to speak specific commands. For example, the user could ask for their schedule in several different ways, such as, but not limited to, “What is my schedule today?”, “Tell me about my day?”, “Do I have anything scheduled for today?”, and “What am I doing today?” The chatbot would then receive and parse the question and generate a response with the user's schedule for today.

The ECSP application may be configured to passively assist in coordinating care for the user between the caregivers. For example, if the caregivers mostly have the care schedule of the user figured out and scheduled, the chatbot may be configured to monitor what the users and caregivers input into the chatbot and provide assistance if necessary. For instance, if one caregiver inputs into the chatbot that the caregiver is taking the user to an appointment on Monday at 2 p.m., the chatbot may respond to the caregiver that the appointment is on their calendar. If another caregiver says that the caregiver is taking the user to breakfast on Tuesday at 10 a.m., the chatbot may respond to the caregiver that the event is not in their calendar and ask the caregiver if the caregiver would like the event added to their calendar. If the caregiver responds that the caregiver would like the event added to their calendar, the chatbot may cause the event to be added to the calendar of the caregiver.

The user and caregivers may also ask the ECSP application questions (e.g., through the chatbot), and the ECSP application may, for example, convert the natural-language question of the user and caregivers into a query, run the query against a database (e.g., an event database stored in a memory device), and transmit a response to the question to the processor including an answer to the question, in response to the query returning the at least one event. For example, the user may ask the ECSP application who is taking them to a haircut appointment or oil change appointment, and the caregiver may ask the ECSP application to identify the last time the user had a bath.

The ECSP application may also notify and/or send alerts to the user and caregivers based upon the user and caregiver alert preferences. For example, the ECSP application may notify a caregiver that a user has not yet taken their medicine, and the ECSP application t may ask the caregiver if the caregiver would like the ECSP application to send a reminder to the user to take their medicine (e.g., through an audible alert, such as via a chatbot). If the caregiver says yes, the ECSP application may automatically cause the reminder to be sent to the user.

In the exemplary embodiment, the ECSP application may further be configured to learn from the user and caregiver requests, responses, and/or questions. For example, if the ECSP application often notifies a caregiver that the user forgets to take a nightly dose of medication, and the caregiver typically tells the ECSP application to remind the user to take their medication in response to the notification from the ECSP application, the ECSP application may automatically cause the ECSP computing device to start reminding the user to take their nightly medicine dosage without input from the caregiver.

Further, the ECSP application may be configured to verbally explain scheduled events, scheduling conflicts, and/or missed scheduled events that may arise to the user and/or caregivers. For example, if the ECSP application determines that a scheduling conflict has arisen (e.g., the caregiver and/or the user are double-booked), the ECSP application may verbally engage with the user and/or caregiver to explain the scheduling conflict. In verbally engaging with the user and/or caregiver, the ECSP application may be configured to converse with the user and/or caregiver to resolve the scheduling conflict. Further, if the ECSP application determines that a scheduled event was missed, the ECSP application may verbally alert the user and/or caregiver of the missed event. In verbally alerting the user and/or caregiver, the ECSP application may also be configured to converse with the user and/or caregiver to resolve and/or reschedule the missed event.

In one exemplary embodiment, the ECSP server may be configured to use the ECSP application to facilitate engagement from the user. In particular, the ECSP server may leverage the ECSP application to encourage interaction and “check-ins” by the user. For example, the ECSP application may provide a daily interactive user interface to the user, may prompt the user to check-in proactively (e.g., through providing a prompt that the user answers), and/or may determine that the user has not checked-in and respond in a reactive manner (e.g., by notifying one or more caregivers that the user has not checked-in in a certain amount of days). As described herein, the daily interactive user interface may include any scheduled activities the user has in their calendar. In addition, the daily interactive user interface may display pictures or content provided by one or more caregivers. For example, a caregiver may provide a picture or article for display within the daily interactive user interface. The ECSP application may further provide an interaction prompt to the user that encourages the user to interact therewith. The interaction prompt may be visual, such as encouraging the user to “tap” a picture or to access a content item (e.g., read an article). The interaction prompt may additionally or alternatively be an audio prompt. For example, the audio prompt may be a question posed to the user (e.g., “How are you feeling today?”) or may be related to a past or future scheduled activity (e.g., “Did you enjoy your Garden Club meeting yesterday?”, “Are you looking forward to seeing the kids for dinner?”). The interaction prompt may additionally or alternatively encourage the user to perform an activity (e.g., “Why don't you take a five-minute walk around?”) The ECSP server may leverage sensor data (e.g., from a wearable device or camera) to determine whether the user completes the suggested activity.

The ECSP server may then transmit messages to a caregiver that provide information about whether and how the user is responding to the interaction prompts. For example, the ECSP server determines whether the user responded to the interaction prompt and includes an indication of any response in daily messages to the caregiver. In this way, the caregiver may be assured that the user is in a positive physical and/or mental state. If the user does not respond to the interaction prompts for a threshold number of days (e.g., two days), the ECSP server may transmit an alert to the caregiver. The alert includes an indication that the user has not responded to interaction prompts for the threshold number of days, which may indicate that the user is hurt, confused, or otherwise in need of a more personal check-in.

The ECSP server may further be configured to generate caregiver analytics, and the ECSP application may be configured to display the generated analytics to the user and caregivers. The ECSP server may generate activity hour, effort hour, and task distribution analytics for each caregiver and compare the analytics to the other caregivers. For example, the ECSP server may generate a chart of the time each caregiver spends caring for the user and/or the time each caregiver spends putting in effort to the care of the user for a predetermined period of time. The ECSP server may further generate a chart of a percentage of tasks for the user that each caregiver handles over the predetermined period of time.

### Exemplary Care Coordination Support System

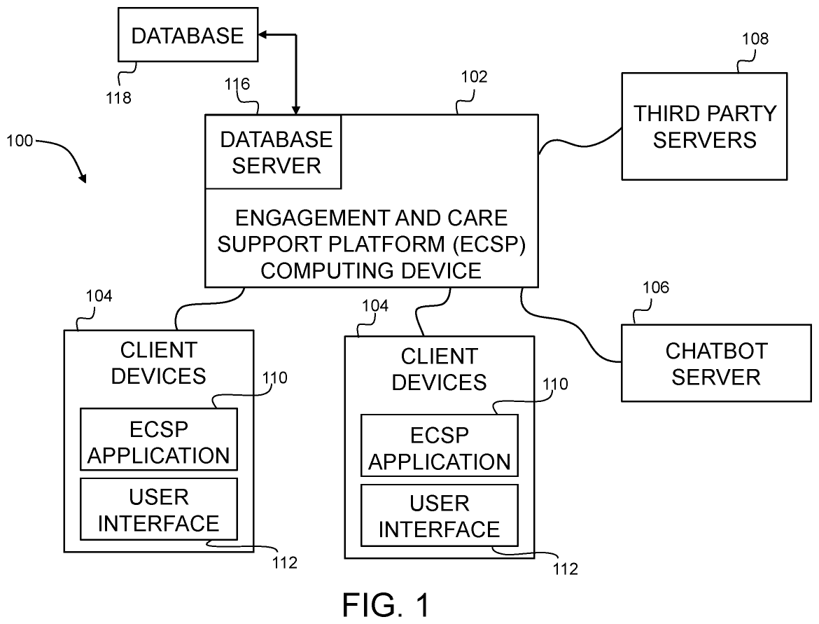

FIG. 1 depicts a view of an exemplary engagement and care support platform (“ECSP”) system 100 that may be used in facilitation engagement of a user and coordinating care of the user between caregivers associated with the user. ECSP system 100 may include a care coordination support platform (“ECSP”) computing device 102. In the exemplary embodiment, ECSP computing device 102 is in communication with client devices 104, a chatbot server 106, and third party servers 108. ECSP computing device 102 is also in communication with a database 118 and may communicate with database 118 through a database server 116.

In some embodiments, database server 116 is a component of ECSP computing device 102. In other embodiments, database server 116 is separate from ECSP computing device 102. In some embodiments, ECSP system 100 may include a plurality of ECSP computing devices 102, client devices 104, third party servers 108, and/or databases 118.

In the exemplary embodiment, ECSP computing device 102 may be configured to store user and caregiver data, generate and/or store a care schedule for the user, and facilitate user engagement with the care schedule (e.g., by prompting the user to check in daily with the caregivers, displaying the care schedule in a user-friendly way, allowing the user to interact with the care schedule, etc.). ECSP computing device 102 may receive user and caregiver data from client devices 104 and use the user and caregiver data to register users and caregivers and generate care schedules for the user and caregivers. For example, a user and a caregiver may download an ECSP application 110 to a device (e.g., client device 104) and input data into ECSP application 110 for registration with a service provided by ECSP computing device 102. The user and caregivers may also access a website of ECSP system 100 using a web browser, and input user data into the website to register with ECSP system 100.

The user data may include personal data (e.g., name, birthdate, height, weight, etc.), user tasks (e.g., taking medicine, bathing, eating, paying bills, getting groceries, car maintenance, home maintenance, etc.), user activities (e.g., social activities, like bingo and golfing, physical activities, like working out and keeping active, etc.), user appointments (e.g., recurring appointments like yearly physicals and bimonthly haircuts, etc.), and any other information associated with the user that may be useful to ECSP computing device 102.

The caregiver data may include personal information (e.g., name, contact information, relationship to the user, role in caring for the user, etc.), caregiver schedule information (e.g., known work and/or activity schedules of the caregivers), caregiver preferences (e.g., which events the caregiver prefers to assist the user with), and any other information associated with the caregivers that may be useful to ECSP computing device 102.

ECSP application 110 may also receive other data from the user and caregivers including notification preferences of the user and caregivers (e.g., preferences of when the user and caregivers would like to be notified and how the user and caregivers would like to be notified, such as receiving a text notification and/or a push button notification from ECSP application 110).

In the exemplary embodiment, users and caregivers may update the user and caregiver data at any time through ECSP application 110. For example, user data that may need to be updated may include a change in and/or newly scheduled events of the user, and a change in a daily medication schedule of the user. For example, caregiver data that may need to be updated may include a change in and/or a new availability schedule of the caregiver and a new activity scheduled by the caregiver.

ECSP application 110 may be in communication with other applications of client device 104 and may import user and caregiver data from the other applications. For example, caregivers may allow ECSP application 110 to retrieve data from a calendar application of the caregivers such that the caregivers may only need to update the schedule associated with the caregiver in one application (e.g., a calendar application).

In the exemplary embodiment, ECSP computing device 102 may be configured to process all of the user and caregiver data ECSP computing device 102 receives from the user and caregivers (e.g., through ECSP application 110) and coordinate a care schedule of the user between the caregivers and facilitate user engagement in the care schedule. In the exemplary embodiment, the caregivers may manually assign tasks to themselves and/or other caregivers through ECSP computing device 102, and ECSP computing device 102 may store the assigned tasks of the care schedule for each caregiver. In some embodiments, ECSP computing device 102 may automatically assign each task, activity, and/or appointment of the user to the caregivers based upon the received caregiver data.

In the exemplary embodiment, client devices 104 may be computers that include a web browser or a software application, which enables client devices 104 to access remote computer devices, such as ECSP computing device 102, using the Internet or other network. More specifically, client devices 104 may be communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem. Client devices 104 may be any device capable of accessing the Internet including, but not limited to, a desktop computer, a laptop computer, a personal digital assistant (PDA), a cellular phone, a smartphone, a tablet, a smart home device (e.g., AMAZON ALEXA, AMAZON ECHO, GOOGLE HOME, and/or RING doorbells), a phablet, wearable electronics (e.g., LIFE ALERT and/or FITBIT), smart watch (e.g., APPLE WATCH), or other web-based connectable equipment or mobile devices.

Further, ECSP computing device 102 may be communicatively coupled to client devices 104 and may receive information from client devices 104. Client device 104 associated with the user may be different from client device 104 associated with the caregiver. For example, client device 104 associated with the user may be a smart home device (e.g., AMAZON ALEXA) because the user may prefer interacting with client device 104 through audio commands rather than physically interacting with client device 104. Client device 104 associated with the caregiver may be a smartphone.

In the exemplary embodiment, some client devices 104 include ECSP application 110 and a user interface 112. User interface 112 may be used, for example, to receive notifications from ECSP computing device 102 and/or to input and verify information to be sent to ECSP computing device 102. ECSP application 110 may be, for example, a program or application that runs on client device 104. Further, ECSP application 110 associated with the user client device 104 may have different functionality as ECSP application 110 associated with the caregiver client devices 104, as is explained in further detail herein, especially with regard to the screenshots of the ECSP application 110.

ECSP computing device 102 may be configured to facilitate user engagement with the care schedule and the caregivers. For example, ECSP computing device 102 (e.g., through ECSP application 110) may display the daily care schedule of the user such that the user is made aware of, and can interact with, their care schedule. ECSP computing device 102 may prompt the user to check-in with the caregivers through ECSP computing device 102. For example, ECSP computing device 102 may display a “Check-In” box that the user may press so that the caregivers know that the user is doing okay and has interacted with their care schedule. For further example, ECSP computing device 102 may keep track of how often the user interacts with ECSP computing device 102 instead of, or in addition to, the user manually checking in. Accordingly, the caregivers are assured that the user is okay, and the user plays an active part in their care schedule.

In some embodiments, ECSP computing device 102 may be configured to display (e.g., through ECSP application 110) the generated care schedule to the user and/or caregivers. ECSP computing device 102 may display the generated care schedule to the user and caregivers through task lists, graphs, calendars, and any other suitable interface that allows the user and caregiver to easily take in and interact with the care schedule of the user.

ECSP computing device 102 may be in communication with chatbot server 106 and leverage the chatbot functionality thereof to implement at least some of the functionality disclosed herein (e.g., to transmit information to and/or receive information from a user and/or one or more caregivers).

Database server 116 may be communicatively coupled to database 118 that stores data. In one embodiment, database 118 may include user data, caregiver data, device data, mobile device data, assignment data, and notification data. In the exemplary embodiment, database 118 may be stored remotely from ECSP computing device 102. In some embodiments, database 118 may be decentralized. In the exemplary embodiment, a user and/or caregiver, may access database 118 via their respective client devices 104 by logging onto ECSP computing device 102, as described herein.

Third party server 108 may be any third party server that ECSP computing device 102 is in communication with that provides additional functionality of ECSP computing device 102 and/or ECSP application 110. For example, third party server 108 may be servers associated with third parties including online retailers/delivery services (e.g., AMAZON, grocery delivery services, food deliver services flower delivery servicers, etc.), ride sharing services (e.g., UBER and LYFT), and hospital/doctor's offices servers. Because ECSP computing device 102 is in communication with third party server 108, the user and/or caregivers may directly access third party servers 108 through ECSP application 110. For example, if a caregiver wants to order flowers for the user, the caregiver may be able to order the flowers from a third party service (e.g., AMAZON) directly through ECSP computing device 102. In some embodiments, third party server 108 may provide updates to the user and/or caregivers through the ECSP application 110 (e.g., notifying the user that their ride is on their way and/or updating the caregiver on the status of their delivery to the user).

The third party server 108 may also be associated with a grocery delivery service and/or a meal ordering service. The third party server 108 may receive orders from at least one client device 104, either that of the senior user or that of one of the senior's circle of care. The grocery/shopping order may be placed based on a stored grocery/shopping list. The order may include a portion of the items on the grocery/shopping list. More than one grocery delivery service may be contacted for the grocery/shopping order based on the availability of different items and available delivery times.

### Exemplary User Computer Device

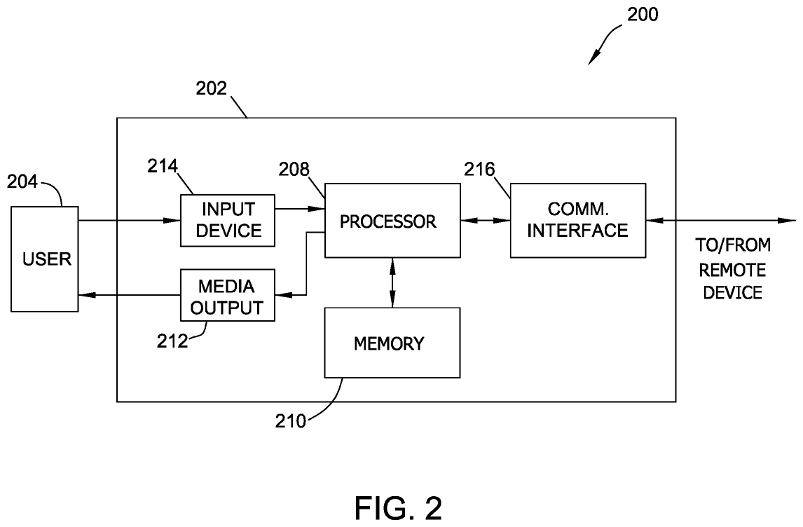

FIG. 2 illustrates an exemplary configuration 200 of an exemplary user computing device 202. In some embodiments, user computing device 202 may be in communication with a care coordination support platform computing device (such as ECSP computing device 102, shown in FIG. 1). User computing device 202 may be representative of, but is not limited to client devices 104 and/or sensor servers 108. For example, user computing device 202 may be a mobile device, smartphone, tablet, smartwatch, wearable electronic, laptop, desktop, or another type of computing device associated with an account holder (e.g., the user and/or the associated caregivers).

User computer device 202 may be operated by a user 204 (e.g., a user of ECSP system 100, shown in FIG. 1 and substantially similar to the user and/or the caregivers described herein). User computer device 202 may receive input from user 204 via an input device 214. User computer device 202 includes a processor 208 for executing instructions. In some embodiments, executable instructions may be stored in a memory area 210. Processor 208 may include one or more processing units (e.g., in a multi-core configuration). Memory area 210 may be any device allowing information such as executable instructions and/or user and registration data to be stored and retrieved. Memory area 210 may include one or more computer-readable media.

User computer device 202 also may include at least one media output component 212 for presenting information to user 204. Media output component 212 may be any component capable of conveying information to user 204 and may be used to at least partially implement user interface 112 (shown in FIG. 1). In some embodiments, media output component 212 may include an output adapter (not shown), such as a video adapter and/or an audio adapter. An output adapter may be operatively coupled to processor 208 and operatively coupleable to an output device, such as a display device (e.g., a cathode ray tube (CRT), liquid crystal display (LCD), light emitting diode (LED) display, or “electronic ink” display) or an audio output device (e.g., a speaker or headphones).

In some embodiments, media output component 212 may be configured to present a graphical user interface (e.g., a web browser and/or a client application) to user 204. A graphical user interface may include, for example, care calendars for the user and/or associated caregivers, lists of assigned tasks of the care schedule for the user and/or caregivers, notifications for the user and/or associated caregivers, assigned tasks of the caregivers, an activity analytics of the caregivers, and/or a messaging page for interacting with the user and/or caregivers. The graphical user interface may additionally include visual interaction prompts that are periodically (e.g., daily, twice daily, every other day, etc.) provided to the user. Visual interaction prompts may include instructions, images, content (e.g., articles or other text), and the like. Media output component 212 may additionally or alternatively provide audible interaction prompts (e.g., via an audio output device).

In some embodiments, user computer device 202 may include input device 214 for receiving input from user 204. User 204 may use input device 214 to, without limitation, interact with ECSP system 100 (e.g., using ECSP application 110), ECSP computing device 102, or any of client devices 104 and third party servers 108 (shown in FIG. 1). Input device 214 may include, for example, a keyboard, a pointing device, a mouse, a stylus, and/or a touch sensitive panel (e.g., a touch pad or a touch screen) and may be used to at least partially implement user interface 112 (shown in FIG. 1). In further embodiments, the input device 214 may be one or more microphones that receive audible commands and/or instructions from the user 204. In these embodiments, the input device 214 receives audio signals from the user 204 and the processor 208 translates those audio signals into text to be acted upon. The processor 208 may also generate replies to the user 204. In these embodiments, the media output component 212 may include one or more speakers that allow the user computer device 202 to communicate to the user 204 with audible verbal communication. The media output component 212 outputs the replies to the user 204. A single component, such as a touch screen, may function as both an output device of media output component 212 and input device 214. User computer device 202 may further include at least one sensor, including, for example, a gyroscope, an accelerometer, a position detector, a biometric input device, and/or an audio input device. In some embodiments, at least some data collected by user computer device 202 may be transmitted to ECSP computing device 102. In the exemplary embodiment, data collected by user computer device 202 may be included in user and caregiver data.

User computer device 202 may also include a communication interface 216, communicatively coupled to any of ECSP computing device 102, client devices 104, and third party servers 108. Communication interface 216 may include, for example, a wired or wireless network adapter and/or a wireless data transceiver for use with a mobile telecommunications network. In some embodiments, the input device 214 receives audio signals from the user 204 and the audio signals are transmitted to a remote computer device via the communication interface 216 for translation and/or interpretation. The communication interface 216 receives an audio response from the remote computer device and plays the audio response through the media output component 212, such as a speaker.

Stored in memory area 210 may be, for example, computer-readable instructions for providing a user interface to user 204 via media output component 212 and, optionally, receiving and processing input from input device 214. The user interface may include, among other possibilities, a web browser and/or a client application. Web browsers enable users, such as user 204, to display and interact with media and other information typically embedded on a web page, a website, or an application hosted by ECSP computing device 102 and/or client device 104. A client application may allow user 204 to interact with, for example, any of ECSP computing device 102, client devices 104, and third party servers 108. For example, instructions may be stored by a cloud service and the output of the execution of the instructions sent to the media output component 212. User computing device 200 may include additional, less, or alternate functionality, including that discussed elsewhere herein.

### Exemplary Server Device

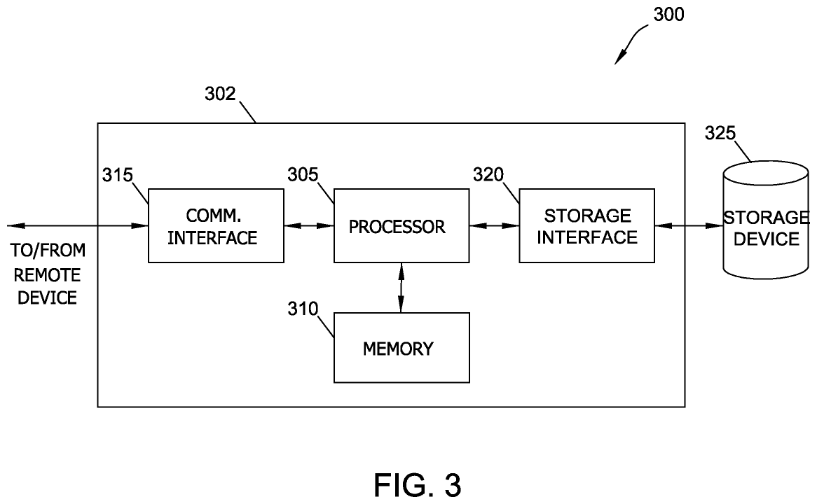

FIG. 3 depicts an exemplary configuration 300 of an exemplary server computer device 302, in accordance with one embodiment of the present disclosure. Server computer device 302 may include, but is not limited to, ECSP computing device 102 (shown in FIG. 1). Server computer device 302 may include a processor 305 for executing instructions. Instructions may be stored in a memory area 310. Processor 305 may include one or more processing units (e.g., in a multi-core configuration).

Processor 305 may be operatively coupled to a communication interface 315 such that server computer device 302 may be capable of communicating with a remote device such as another server computer device 302 or a user computing device, such as client device 104 (shown in FIG. 1). For example, communication interface 315 may receive requests from or transmit requests to client devices 104 via the Internet.

Processor 305 may also be operatively coupled to a storage device 325. Storage device 325 may be any computer-operated hardware suitable for storing and/or retrieving data, such as, but not limited to, data associated with database 118 (shown in FIG. 1). In some embodiments, storage device 325 may be integrated in server computer device 302. For example, server computer device 302 may include one or more hard disk drives as storage device 325. In other embodiments, storage device 325 may be external to server computer device 302 and may be accessed by a plurality of server computer devices 302. For example, storage device 325 may include a storage area network (SAN), a network attached storage (NAS) system, and/or multiple storage units such as hard disks and/or solid state disks in a redundant array of inexpensive disks (RAID) configuration.

In some embodiments, processor 305 may be operatively coupled to storage device 325 via a storage interface 320. Storage interface 320 may be any component capable of providing processor 305 with access to storage device 325. Storage interface 320 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 305 with access to storage device 325.

Processor 305 executes computer-executable instructions for implementing aspects of the disclosure. In some embodiments, processor 305 may be transformed into a special purpose microprocessor by executing computer-executable instructions or by otherwise being programmed.

### Exemplary Computer-Implemented Method

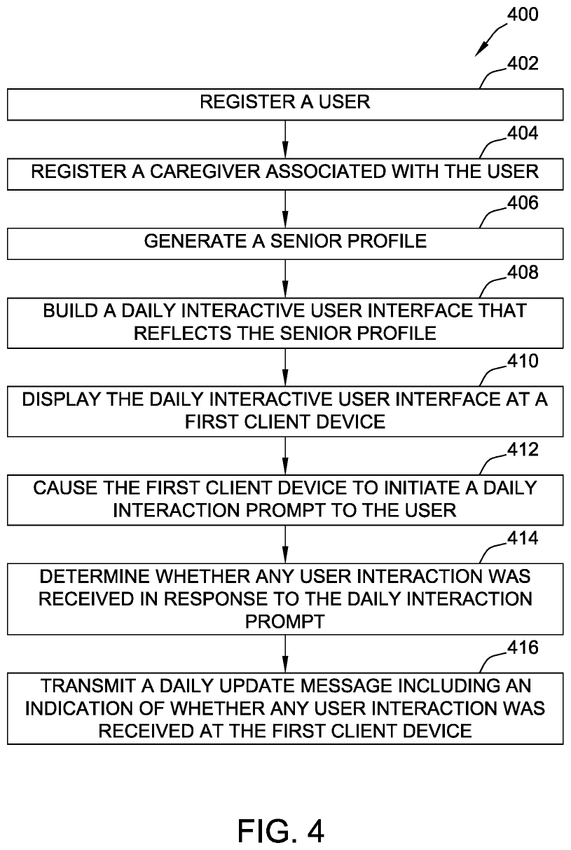

FIG. 4 depicts a flow chart illustrating a computer-implemented method 400 for facilitating engagement of a senior user. In the exemplary embodiment, method 400 may be implemented by a care coordination support platform computer system such as ECSP computing device 102 (shown in FIG. 1).

Method 400 may include registering 402 a user and registering 404 at least one caregiver associated with the user for a care coordination support platform service (e.g., provided by ECSP computer system shown in FIG. 1). Method 400 may also include generating 406 a senior profile based upon user personal and scheduling data provided during registering 402. Method 400 may further include building 408 a daily interactive user interface that reflects the senior profile.

Method 400 may also include displaying 410 the daily interactive user interface at a first client device associated with the user, for example, via an application associated with ECSP computing device 102, shown in FIG. 1. Method 400 may further include causing 412 the first client device to initiate a daily interaction prompt to the user, and determining 414 whether any user interaction was received at the first client device in response to the daily interaction prompt. In some embodiments, the daily interaction prompt may be a good morning message that is displayed on a display screen. In other embodiments, the daily interaction prompt may be an audible, verbal good morning message that is output through one or more speakers to the user when the user activates the ECSP application. In other embodiments, the ECSP computing device 102 may include at least one microphone and detect movement of the user nearby by monitoring the audio signals collected by the at least one microphone. The ECSP computer device 102 may then determine which speaker is closest to the user based on where the noises of movement are detected. Then the ECSP computer device 102 instructs that speaker to transmit the daily interaction prompt.

In addition, method 400 may include transmitting 416 a daily update message to a second client device associated with the caregiver, the daily update message including an indication of whether any user interaction was received at the first client device. The daily update message may also include a summary of the user's interactions with the first client device. The summary of the user's interactions may be a description of the interaction, such as “The user asked about the weather.” The summary can also include a genericized description of the interaction, such as “10:42 AM Entertainment Music” or “12:38 PM Weather.” The genericized version of the summary message allows the senior user to keep their privacy, while allowing the caregivers to have an idea of what the senior user is doing, but without any details. In another version of the summary message, the second client device may receive a message that the senior user performed a search, but not what the search was for or about. In some embodiments, the summary message may be sent to the second client devices every time that the senior user interacts with the first client device. In other embodiments, the summary message may be sent on a periodic basis, such as, but not limited to, once an hour, once a day, etc. The period may be based on one or more preferences set by either the senior user and/or the caregiver.

### Exemplary Computer Device

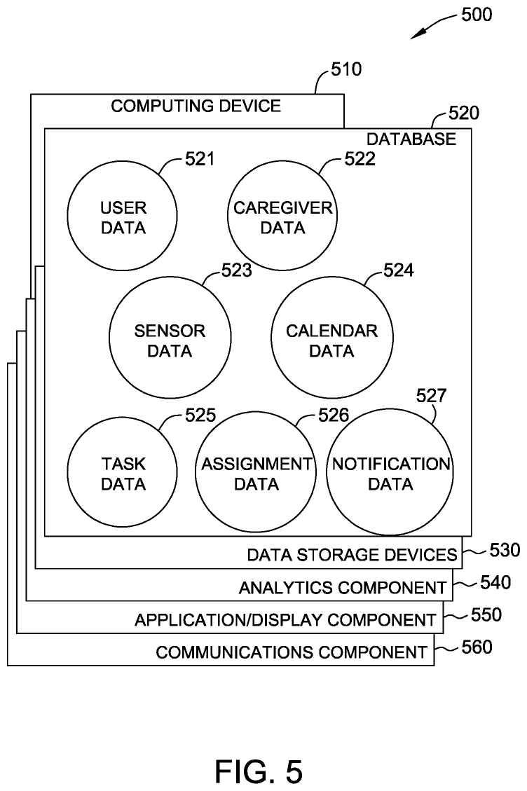

FIG. 5 depicts a diagram 500 of components of one or more exemplary computing devices 510 that may be used in care coordination support platform system 100 (shown in FIG. 1). In some embodiments, computing device 510 may be similar to ECSP computing device 102 (shown in FIG. 1). Database 520 may be coupled with several separate components within computing device 510, which perform specific tasks. In this embodiment, database 520 may include user data 521, caregiver data 522, sensor data 523, calendar data 524, task data 525, assignment data 526, and notification data 527. In some embodiments, database 520 is similar to database 118 (shown in FIG. 1).

Computing device 510 may include database 520, as well as data storage devices 530. Computing device 510 may also include an analytics component 540 for analyzing received user data to generate a senior profile based upon the received data. The senior profile may be used to recommend activities, provide content of interest (e.g., articles), track schedules, and the like. Analytics component 540 may be further configured to analyze received data to determine whether a user has responded to an interaction prompt, as described herein. Computing device 510 may further include application/display component 550 for generating and displaying information (e.g., interaction prompts) to users, such as through ECSP application 110 (shown in FIG. 1), and supporting ECSP application 110. Moreover, computing device 510 may include communications component 560 for receiving and transmitting data (e.g., to and from client devices 104), such as user data 521, caregiver data 522, sensor data 523, calendar data 524, task data 525, assignment data 526, and notification data (e.g., daily update messages) 527, as well as responses to interaction prompts. Computing devices 510 may include additional, less, or alternate functionality, including that discussed elsewhere herein.

### Exemplary Care Coordination Support Application

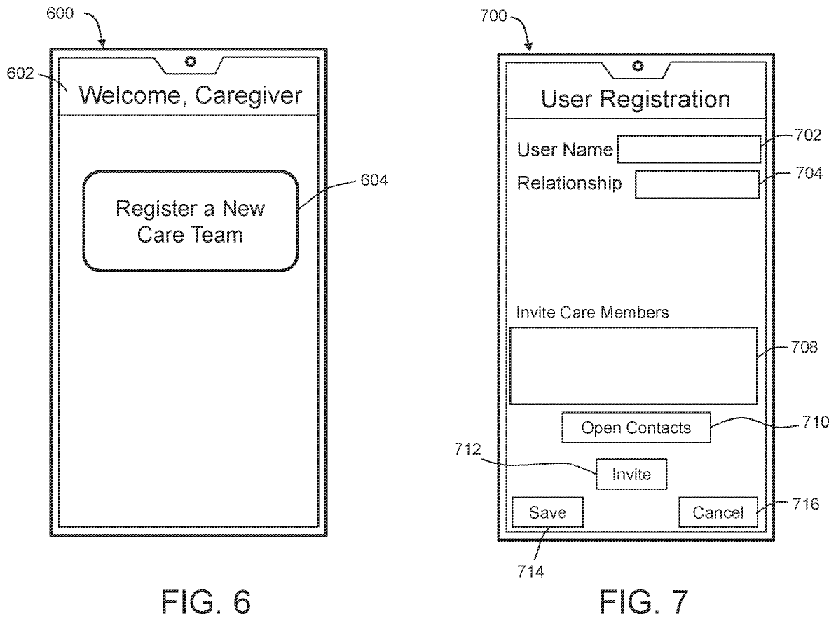

FIGS. 6-18 include screenshots of one example embodiment of an application (e.g., ECSP application 110, shown in FIG. 1) executable as part of an engagement and care support platform system (e.g., engagement and care support platform system 100, shown in FIG. 1). The application may be accessible on any suitable electronic device, such as a mobile phone, tablet, smart home device, watch, or any other computing device. The application enables a user to check in and interact with the application, determine what tasks caregivers need to complete, view a care schedule of the user, and enables one or more caregivers to add tasks for the user, view the tasks that the one or more caregivers have to complete for and/or with the user, and coordinate the care schedule between the one or more caregivers.

In some embodiments, the application may enable the user and the one or more caregivers to subscribe to alerts, notifications, and/or reminders.

The application may be configured to communicate with various other software and/or applications on the computing devices of the users and/or the one or more caregivers. For example, the application may be able to access or otherwise communicate with calendar applications and/or contact applications. The application may be configured to retrieve data from and/or report data to these other applications. In addition, the application may be configured to track, monitor, and/or record application utilization metrics for the user and/or the caregivers, such as how often the user and/or the caregivers access the application, and the various features of the application used by the user and/or the caregivers.

In one embodiment, the application, once downloaded onto the computing device of the user and/or the caregivers, may not require internet connectivity to perform some or all of the functionality of the application (e.g., setting alerts and notifying the user and/or caregivers of the alerts). In some embodiments, all or a portion of the data input by the user and/or caregivers into the application (including, for example, application utilization metrics, task logs, etc.) may be electronically transmitted to a server (e.g., ECSP server 102) for processing, and the processed data may be transmitted back for further processing and/or display by the application.

In the exemplary embodiment, the application can be configured to, inter alia, allow the user to quickly and easily check-in with the caregivers, giving the caregivers peace of mind, proactively allowing the user to check-in (e.g., by prompting the user to answer a question like “How are you feeling today?”), providing a reactive response if/when the user does not check in (e.g., by notifying one or more of the caregivers), providing an interactive display for the user and the caregivers, provide tools for the user and the caregivers to coordinate key tasks associated with the care schedule of the user, providing smart suggestions of media for the user to increase user engagement with the application, allowing caregivers to easily share photos and videos with the user, allowing the user to easily view the shared photos and videos, allowing the user to view their care schedule with audio commands and/or through interacting with the digital display of the application, and providing social features that help the user and caregivers stay and feel connected.

In some embodiments, the application transmits the prompt to the user via audio signals transmitted to the media output component 212 (shown in FIG. 2), such as a speaker. The application receives the response through an input device 214 (shown in FIG. 2), such as a microphone. In some embodiments, the input device 214 receives audio signals from the user 204 and the audio signals are transmitted to a remote computer device via the communication interface 216 for translation and/or interpretation. The communication interface 216 receives an audio response from the remote computer device and plays the audio response through the media output component 212, such as a speaker.

FIG. 6 illustrates an initial welcome page 600 that may include a header 602. Although not specifically shown, header 602 may include a home button, a back button, and any other buttons to help the user and/or the caregivers navigate the application. In some embodiments, initial welcome page 600 may further include a footer (not specifically shown) that may include additional buttons to help the user and/or the caregiver navigate the application. Initial welcome screen 600 may also include a “Register a New Care Team” button 604 that, when clicked, may cause the application to display a user registration screen 700.

FIG. 7 illustrates user registration page 700 for the application. User registration page 700 may include a first field 702 for the user and/or an administrative caregiver (e.g., “admin caregiver”) to enter the name of the user and a second field 704 to, if user registration page 700 is filled out by the admin caregiver, enter the relationship of the admin caregiver to the user.

Further, user registration page 700 may include a third field 708 for the user and/or admin caregiver to enter email addresses, phone numbers, and/or other contact information of other caregivers such that the application may invite the other caregivers to download the application. User registration page 700 may include a button 710 that, when clicked, may cause the application to open a contacts list of the user such that the user can choose contacts to invite instead of manually filling out third field 708.

User registration page may include an “Invite” button 712, that, when clicked, may cause the application to invite the other caregivers to download the application and/or show a caregiver registration page 800, a “Save” button 714 that, when clicked, may cause the application to save the information in fields 702, 704, and 708, and a “Cancel” button 716 that, when clicked, may cause the application to go back to initial welcome page 600. Once other caregivers download the application, the other caregivers may be directly navigated to caregiver registration page 800 and may bypass initial welcome page 600 and/or user registration page 700.

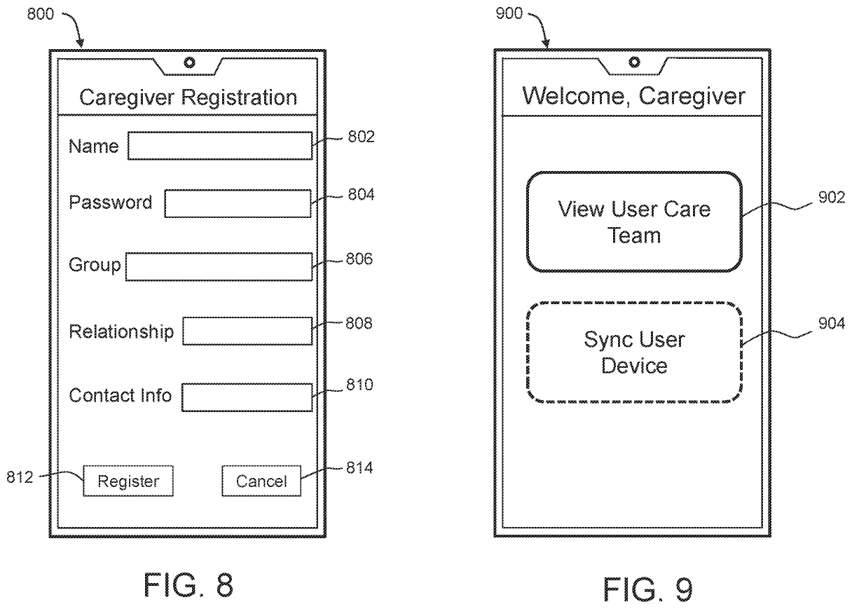

FIG. 8 illustrates caregiver registration page 800 for the application that allows the admin caregiver and/or the other caregivers to register for the application. Caregiver registration page 800 may include a first field 802 for the caregiver to enter the name of the caregiver, a second field 804 for the caregiver to enter a password for the application, a third field 806 for the caregiver to enter a group that the caregiver belongs to (e.g., “Kids,” “Grandkids,” etc.), a fourth field 808 for the caregiver to enter a relationship of the caregiver to the user, and a fifth field 810 for the caregiver to enter contact information (e.g., phone number(s), email address, and/or home address).

Caregiver registration page 800 may further include a “Register” button 812 that, when clicked, may cause the application to save and store the information that caregiver entered into fields 802, 804, 806, 808, and/or 810, and display a welcome screen 900, and caregiver registration page 800 may include a “Cancel” button 814 that, when clicked, may cause the application to close and show a different screen (e.g., a home screen) of the device running the application.

FIG. 9 illustrates welcome page 900 that may be the first page that the user and/or the admin caregiver are directed to until a user care team is complete (e.g., the user and all caregivers associated with the user are registered through the application). Welcome page 900 may include a “View User Care Team” button 902 that, when pressed, may cause the application to display a user care team page 1000. Welcome page 900 may further include a “Sync User Device” button 904 (e.g., if the device of the user has not been synced with the application) that, when pressed, may cause the application to display instructions for syncing the user device with the application (not specifically shown).

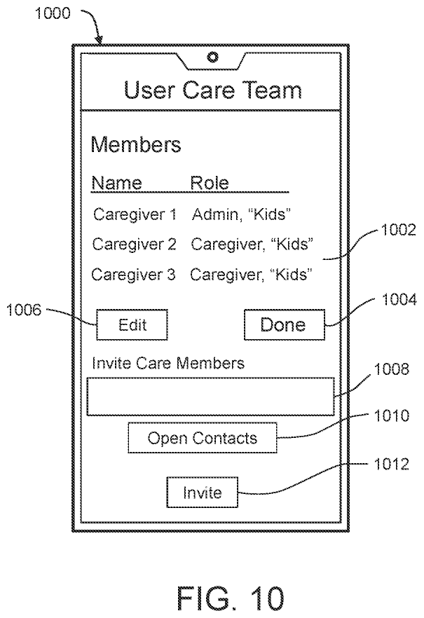

FIG. 10 illustrates user care team page 1000 that may allow the user and/or the admin caregiver to view and/or edit the care team associated with the user. User care team page 1000 may include a list 1002 of registered members of the care team including the names of the caregivers (e.g., entered by the caregivers in first field 802 of caregiver registration page 800, shown in FIG. 8) and groups of the caregivers (e.g., entered by the caregivers in third field 806 of caregiver registration page 800, shown in FIG. 8).

User care team page 1000 may also include an “Edit” button 1006 that, when clicked, allows the user and/or admin caregiver to edit list 1002 and a “Done” button 1004 that, when clicked, causes the application to save the care team and store the care team as fully registered. User care team page 1000 may also include a first field 1008 where the user and/or the admin caregiver may manually enter contact information of additional caregivers to add to the care team.

Additionally or alternatively, the user and/or the admin caregiver may press a “Open Contacts” button 1010 that, when pressed, causes the application to open the contacts of the user and/or admin caregiver and allow the user and/or admin caregiver to automatically choose which contacts the user and/or admin caregiver would like to invite to the care team. When the user and/or admin caregiver is done adding contact information of additional caregivers, the user and/or admin caregiver may press an “Invite” button 1012 that, when clicked, causes the application to invite the additional caregivers to register for the application.

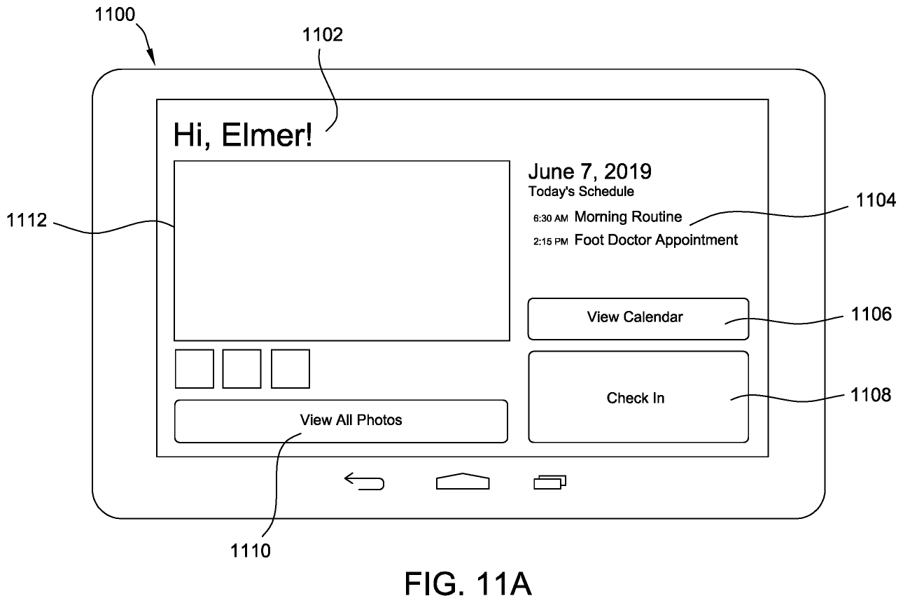

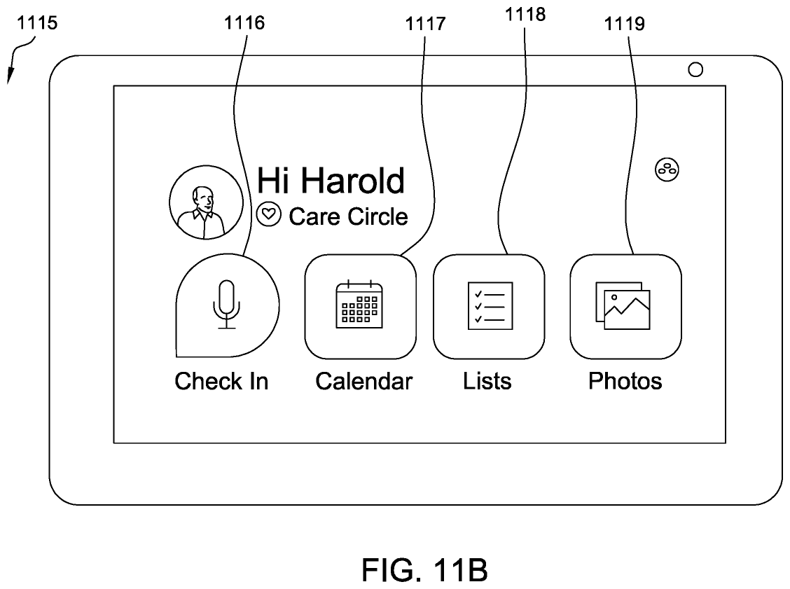

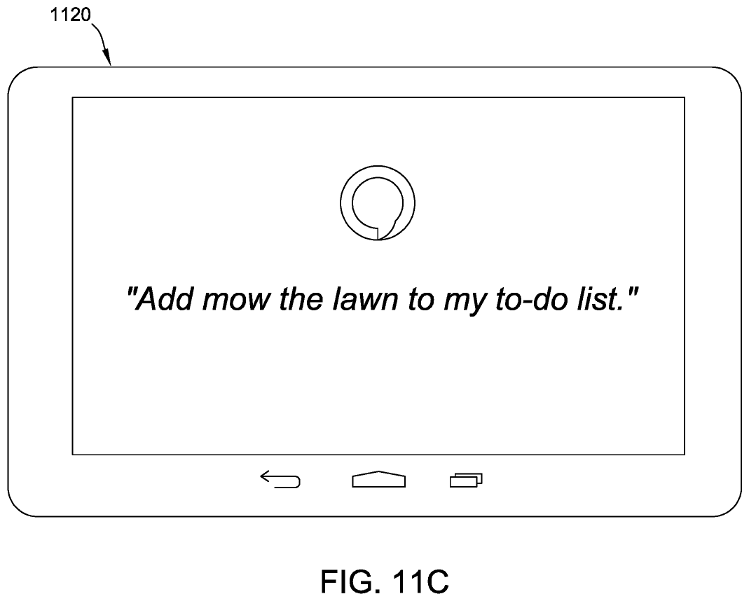

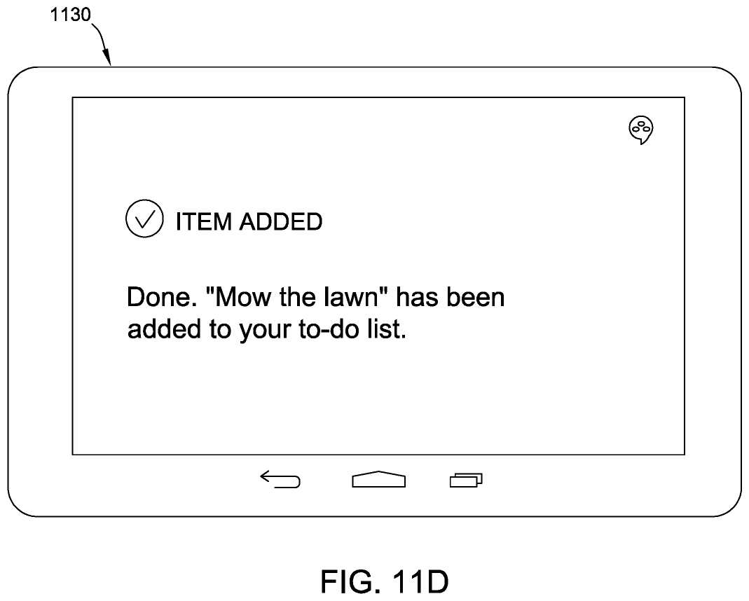

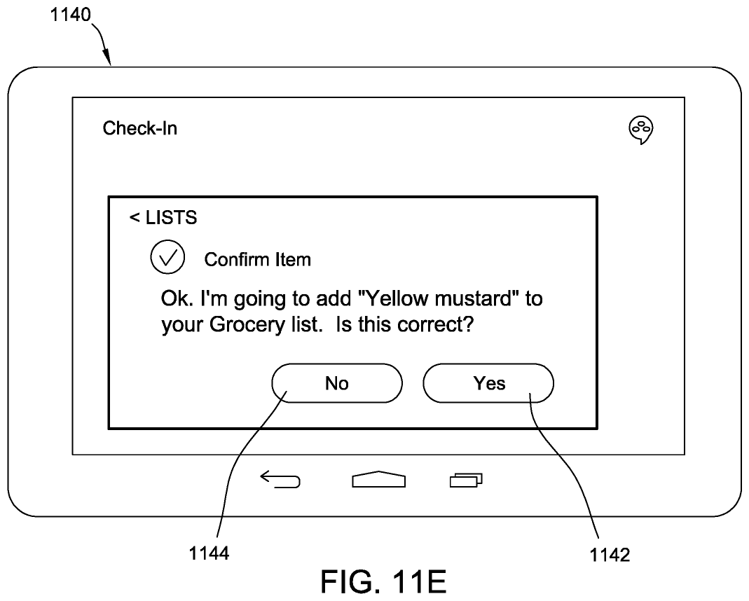

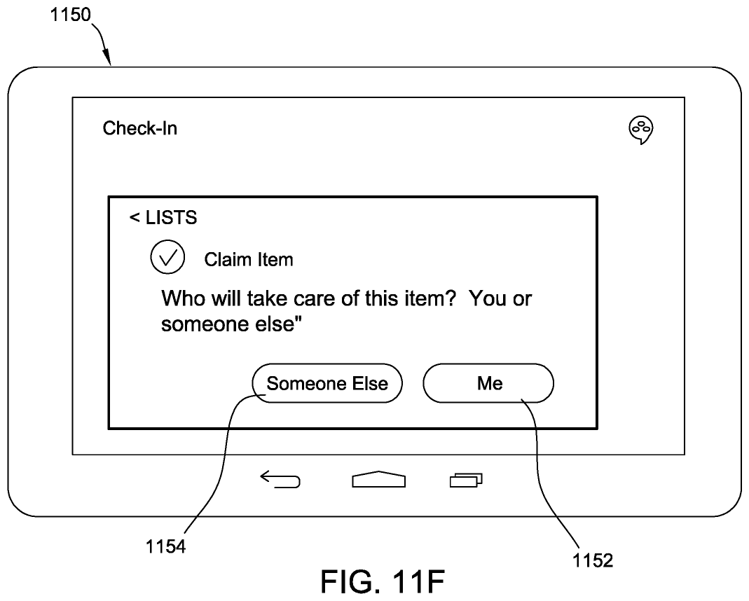

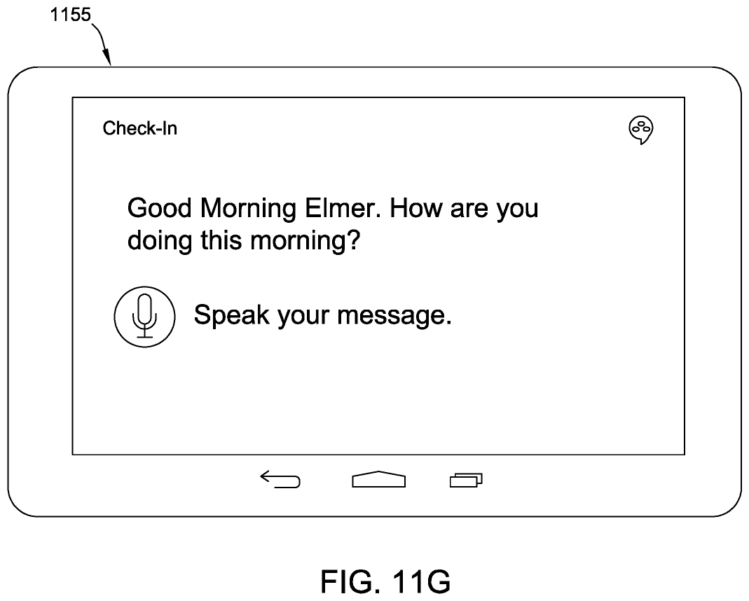

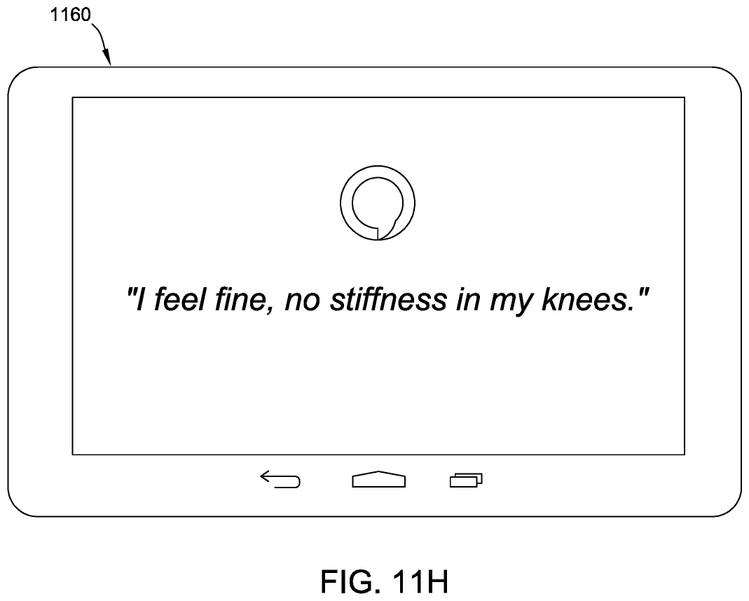

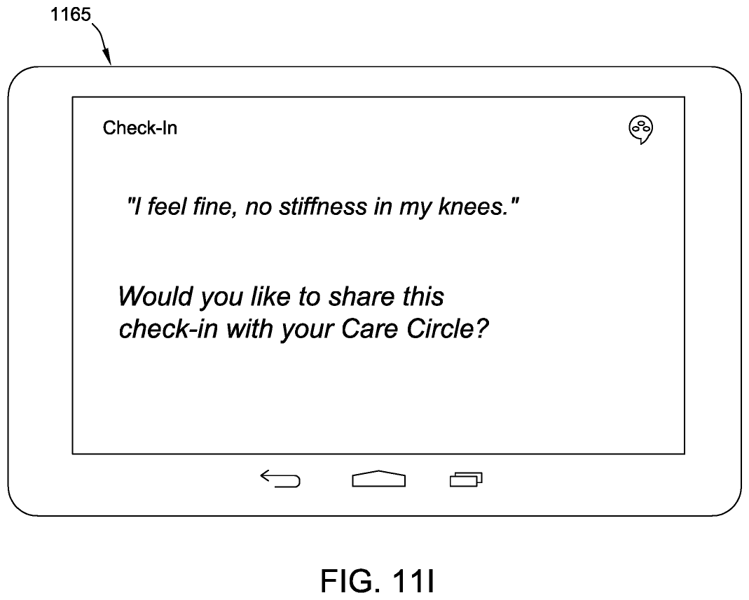

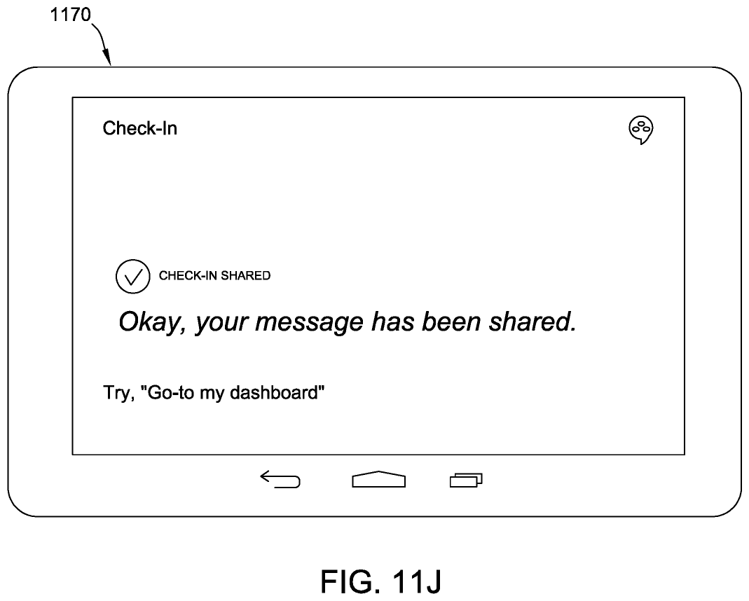

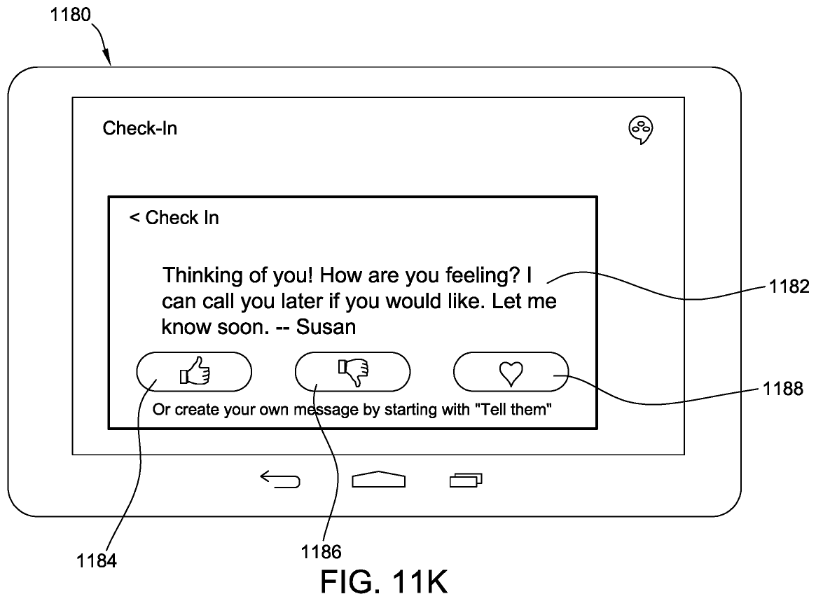

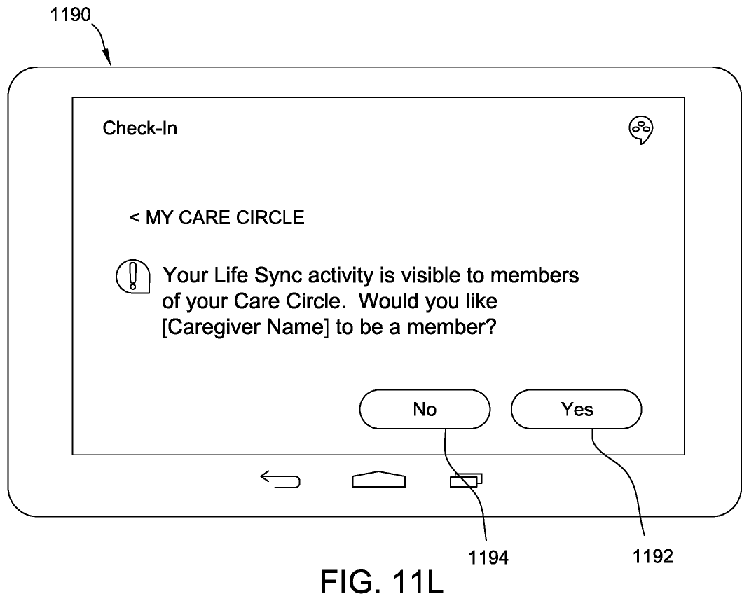

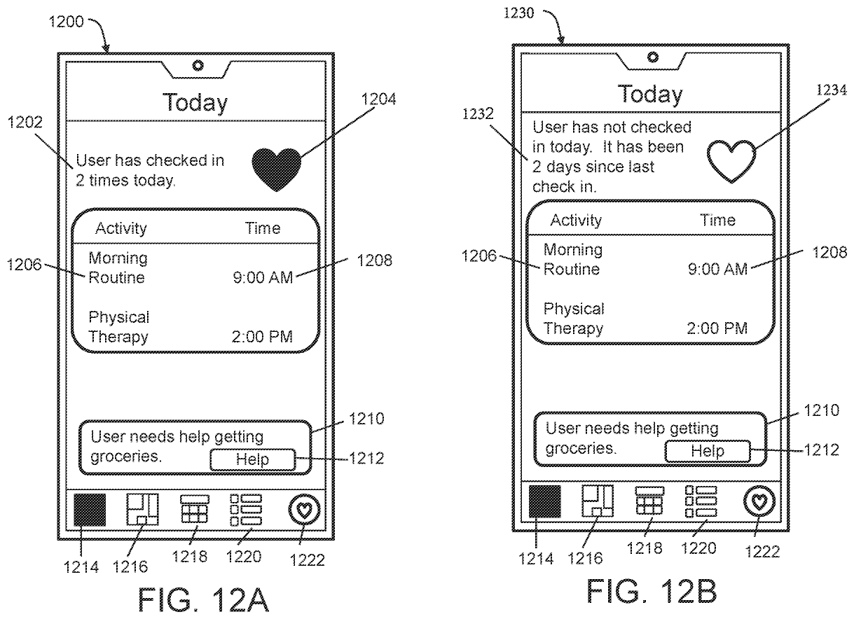

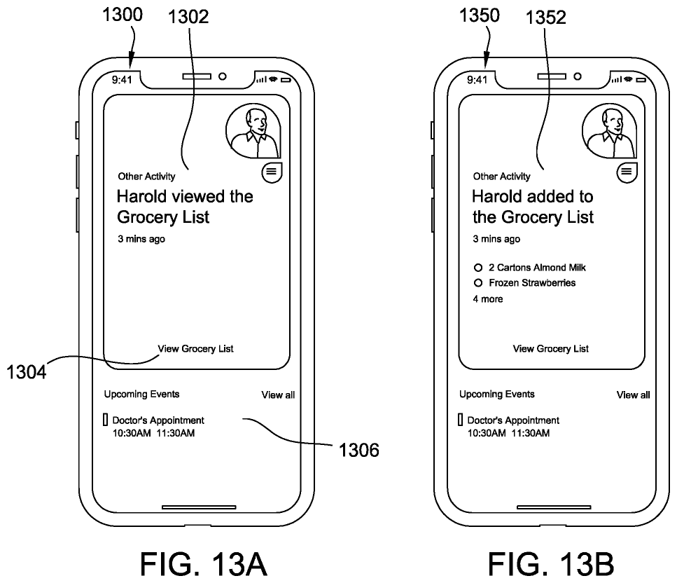

FIG. 11A shows an example user home page 1100 displayed on a device associated with the user that the user may be directed to when the device is synced with the application. User home page 1100 may include a welcome greeting 1102 and a list 1104 of daily tasks scheduled for the user. User home page 1100 may further include a “View Calendar” button 1106 that, when pressed, may cause the application to display a detailed calendar of the user (not specifically shown). User home page 1100 may further include a “Check In” button that, when pressed, may notify the caregivers (e.g., in message 1202 and 1302 of user home screen 1200 and 1300, shown in FIGS. 12 and 13, respectively) that the user has checked in. User home page 1100 may also include media 1112 shared with the user by the caregivers and a “View All Photos” button 1110 that, when pressed, causes the application to display all media 1112 shared with the user.

FIG. 11B shows an alternative user home page 1115 displayed on a device associated with the user that the user may be directed to when the device is synced with the application. User home page 1115 may include a “Check In” button 1116 that, when pressed, functions substantially the same as button 1108 of FIG. 11A. That is, button 1116, when pressed, may notify the caregivers (e.g., in message 1202 and 1232 of user home screen 1200 and 1230, shown in FIGS. 12A and 12B, respectively) that the user has checked in. Home page 1115 may further include a “Calendar” button 1117 that, when pressed, may cause the application to display a screen showing the calendar of the user (e.g., displayed as a daily calendar, a weekly calendar, a monthly calendar, etc.). Home page 1115 may further include a “Lists” button 1118 that, when pressed, may cause the application to display a screen showing lists associated with the user (e.g., a grocery/shopping list, a to-do list, etc.). User home page 1115 may also include a “Photos” button 1119 that, when pressed, may cause the application to display photos shared with the user.

FIGS. 11C-11L show user interaction pages 1120, 1130, 1140, 1150, 1155, 1160, 1165, 1170, 1180, and 1190. User interaction pages 1120 and 1130 are displayed on the application when the user interacts with the ECSP application 110. For example, user interaction page 1120 displays that the user used voice-activation to command the application to “Add mow the lawn to my to-do list.” The user's statement is received as audible speech by an input device 214 (shown in FIG. 2), such as a microphone, which translates the audible speech into audio signals. The ECSP application 110 transmits the audio signals to a chatbot, such as the chatbot server 106 (shown in FIG. 1). The chatbot server 106 may be on the same computing device as the application or the chatbot server 106 may be on a remote computing device. The chatbot server 106 receives and parses the audio signals to determine the audible statement. The chatbot server 106 then generates a response. The response is based on the subject matter of the audible statement. The response is transmitted back as audible signals. The first client device 104 plays the response through the media output component 212 (shown in FIG. 2), such as a speaker. In other embodiments, the response is generated as a display to be displayed on a user interaction page 1130.

User interaction page 1130 displays that the application received the command from the user as shown in user interaction page 1120 and added the command to the to-do list of the user. User interaction page 1140 displays that the user used a command (e.g., either a voice command or pressed button 1118 of user interaction screen 1115) to add “yellow mustard” to their “Grocery/Shopping list.” User interaction page 1140 includes a “Yes” button 1142 that, when pressed, causes the application to add yellow mustard to the grocery/shopping list and display user interaction page 1150, and a “No” button 1144 that, when pressed, causes the application to not add yellow mustard to the grocery/shopping list and return to, for example, user home page 1110 or alternative user home page 1115. User interaction page 1150 includes a prompt for the user about whether the user will handle the newly added grocery/shopping list item or if the user would like somebody else (e.g., a caregiver) to handle the newly added grocery/shopping list item. User interaction page 1150 includes a “Me” button 1152 that, when pressed, causes the application to add the newly added grocery/shopping list item to the lists of the user and then display, for example, user home page 1110 or alternative home page 1115, and a “Somebody else” button 1154 that, when pressed, causes the application to add the newly added grocery/shopping list item to the lists of one of the caregivers of the user. Accordingly, the “Somebody else” button 1154, when pressed, may cause the application to display a list of the caregivers associated with the user (not shown) such that the user can pick the caregiver to assign the task to.

In the exemplary embodiment, ECSP server 102 and/or the ECSP application 110 stores multiple tables for different lists, such as in a database 118 (shown in FIG. 1). Each table includes a plurality of entries for the corresponding list. For example, there may be a grocery/shopping list, a To-do list, and a home repairs list. In some embodiments, the user and/or one of the caregivers may create additional lists, such as, but not limited to, holiday meal shopping list, holiday present shopping list, birthday shopping list, Friday meal grocery/shopping, etc. In these embodiments, the ECSP server 102 and/or the ECSP application 110 may create an additional table for each list.

When the user confirms an item to be added to the list, see step 2010, the ECSP server 102 and/or the ECSP application 110 adds the confirmed item to the corresponding list. In some embodiments, the user may verbally ask the ECSP server 102 and/or the ECSP application 110 to read off the items on a list, such as the grocery/shopping. In these embodiments, the ECSP server 102 and/or the ECSP application 110 accesses the corresponding table and the chatbot server 106 translates each item into audible speech. In these embodiments, the ECSP server 102 and/or the ECSP application 110 transmits the list of items to the chatbot server 106. In other items, the ECSP server 102 and/or the ECSP application 110 transmits one item at a time to the chatbot server 106 to be spoken out loud to the user.

Furthermore, the table may include a plurality of fields for each item on the list, including, but not limited to, when added to the list, who added the item to the list, who is assigned the item on the list, and when the item was removed or marked completed. If an item does not have an individual assigned to it, then the ECSP server 102 and/or the ECSP application 110 may request an individual to be assigned to the item. In some embodiments, the ECSP server 102 and/or the ECSP application 110 verbally asks the user. In other embodiments, the ECSP server 102 and/or the ECSP application 110 transmits one or more notifications to one or more caregivers to see who will volunteer for the item. In some embodiments, the ECSP server 102 and/or the ECSP application 110 finds similar items that have already been completed in the table and determines who was assigned to those item. The ECSP server 102 and/or the ECSP application 110 then asks that individual first to determine if they will volunteer for the current item.

User interaction pages 1155, 1160, 1165, 1170, and 1180 are displayed on the application when the application actively interacts with the user for a check in. For example, user interaction page 1140 displays that the application has prompted the user to check-in via an audio prompt (e.g., asking the user “How are you this morning?”). User interaction page 1160 displays that the user used a voice command to respond to the application that the user is feeling fine (e.g., by saying, “I feel fine. No stiffness in my knees.”).

User interaction page 1165 displays that the application received the response of the user and includes a prompt for the user to decide whether to share the check-in with the caregivers (e.g., the application asks the user, “Would you like to share this check-in with your care circle?”). User interaction page 1170 displays that the application received the response of the user to the prompt of user interaction page 1165 (e.g., the user responded that the user would like to share their check-in with their care circle and the application displays that the check-in has been shared). User interaction page 1180 displays an alternate way for the user to check-in. Specifically, user interaction page 1180 displays a message 1182 from a caregiver asking the user to check in with the caregiver soon. Further, user interaction page 1180 includes three check-in buttons, a first check-in button 1184, a second check-in button 1186, and a third check-in button 1188. The first check-in button 1184, when pressed or commanded to be pressed (e.g., via voice command), causes the application to send a message to the caregiver associated with the message 1182 that the user is doing well today. The second check-in button 1186, when pressed or commanded to be pressed (e.g., via voice command), causes the application to send a message to the caregiver associated with the message 1182 that the user is not doing well today. The third check-in button 1188, when pressed or commanded to be pressed (e.g., via voice command), causes the application to send a message to the caregiver associated with the message 1182 that the user feels loved today. Accordingly, when any of first, second, and third check-in buttons 1184, 1186, and 1188 is pressed or commanded to be pressed, the application counts the user as checked-in for the day.

User interaction page 1190 displays that a caregiver has been added to the Care Circle and prompts the user to accept or deny the user. User interaction page 1190 includes a “Yes” button 1192 and a “No” button 1194. The “Yes” button 1192, when pressed or commanded to be pressed (e.g., through voice command), causes the application to add the respective caregiver to be added to the Care Circle of the user. The “No” button 1194, when pressed or commanded to be pressed (e.g., through voice command), causes the application to deny the respective caregiver access to the Care Circle of the user. Accordingly, the application allows the user to control the members of the Care Circle of caregivers associated with the user.

FIGS. 12A and 12B are examples of caregiver home screens 1200 and 1230. For example, caregiver home screens 1200 and 1230 may be the first screen that is displayed when the caregivers log-in to the application after registering the care team has been completed (e.g., in user care team page 1000). Caregiver home screens 1200 and 1230 may be substantially similar and may include a list 1206 of activities scheduled for the user and times 1208 associated with the activities. Caregiver home screens 1200 and 1230 may further include a notification 1210 of what the user needs help with (e.g., unassigned tasks) and a button 1212 that, when pressed, cause the application to assign the task of notification 1210 to the caregiver.

Caregiver home screens 1200 and 1230 may further include navigation buttons 1214, 1216, 1218, 1220, and 1222 that, when pressed, cause the application to display different screens of the application. For example, pressing button 1214 may cause the application to display one of caregiver home screens 1200 and 1230. Caregiver home screens 1200 and 1230 may further include a message 1202 and 1232, respectively that may be accompanied by a logo 1204 and 1234. For example, when message 1202 and/or 1232 indicates that the user has checked-in for a specific day, logo 1204 and/or 1234 may be filled in (e.g., as shown in logo 1204). When message 1202 and/or 1232 indicates that the user has not checked-in for a specific day, logo 1204 and/or 1234 may not be filled in (e.g., as shown in logo 1234).

FIGS. 13A and 13B are alternative examples of caregiver home screens 1300 and 1350. For example, caregiver home screens 1300 and 1350 may be the first screen that is displayed when the caregivers log-in to the application after registering the care team has been completed (e.g., in user care team page 1000). Caregiver home screens 1300 and 1350 may include an action button 1304 that, when pressed, may cause the application to display a screen related to the action button (e.g., a grocery/shopping list view screen). Caregiver home screens 1300 and 1350 may further include an upcoming events list 1306 that display upcoming events scheduled for and/or associated with the user (e.g., a doctor's appointment scheduled at 10:30 A.M.).

Caregiver home screens 1300 and 1350 may be substantially similar and may include a user activity message 1302 and 1352. User activity messages 1302 and/or 1352 may display the most recent user activity and/or interaction with the application. For example, user activity messages 1302 and/or 1352 may show that the user viewed a grocery/shopping list (e.g., as shown in user activity message 1302) and/or that the user added to the grocery/shopping list and list the items that the user added (e.g., as shown in user activity message 1352).

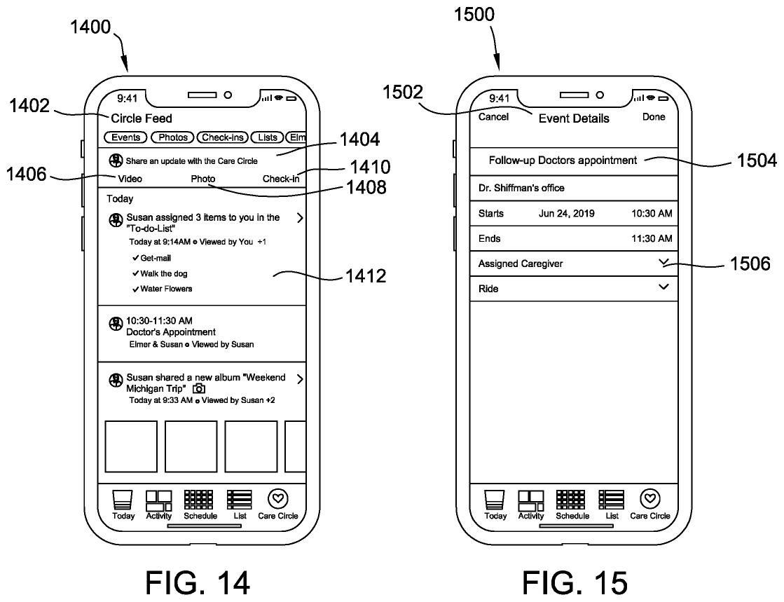

FIG. 14 shows a caregiver feed page 1400 (e.g., which the application displays when button 1216 of FIGS. 12 and 13 is pressed). Caregiver feed page 1400 may include a header 1402, a field 1404 accompanied by a video button 1406, a photo button 1408, and a check-in button 1410, and a caregiver activity list 1412. The caregiver may give updates and include any information that the caregiver wishes to share with the other caregivers in field 1404. Video button 1406, when pressed, allows the caregiver to share a video on the application, photo button 1408, when pressed, allows the caregiver to share a photo (or multiple photos) on the application, and check-in button 1410, when pressed, allows the caregiver to check-in with the other caregivers (e.g., when the caregiver is carrying out an assigned task for the user and/or checking in on the user). Caregiver activity list 1412 shows a feed of recent caregiver activities (e.g., assigned tasks, adding appointments, sharing photos and/or videos, etc.).

FIG. 15 shows a caregiver schedule page 1500 (e.g., which the application displays when button 1218 of FIGS. 12 and 13 is pressed) that allows the caregivers to add events and/or appointments to the care calendar of the user. Caregiver schedule page 1500 may include a header 1502, fields 1504, and pull-down bars 1506. The caregivers may provide information about the new event in field 1504, and the caregivers may use pull-down bars 1506 to assign the event to a specific caregiver.

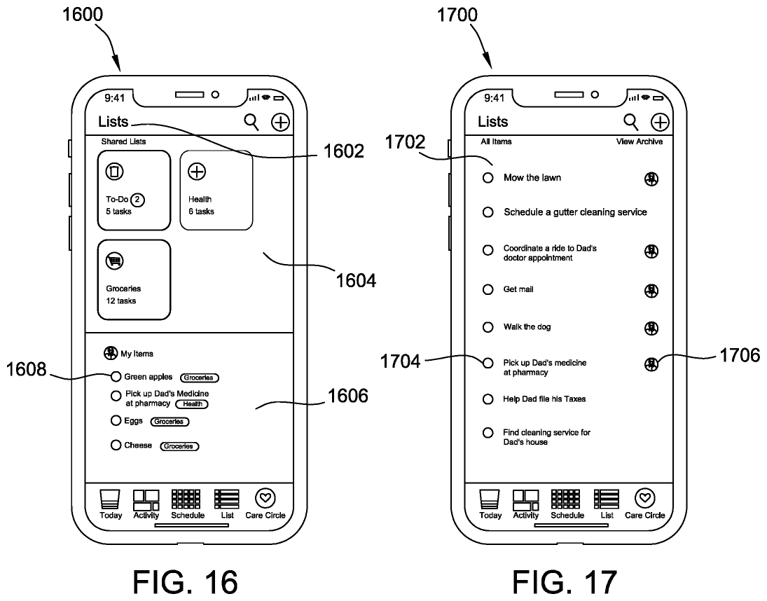

FIGS. 16 and 17 show caregiver list pages 1600 and 1700. Caregiver list page 1600 (e.g., which the application may display when button 1220 of FIGS. 12 and 13 is pressed) may include a header 1602, different categories of lists 1604, and list items 1606 that are assigned to the caregiver. List items 1606 may be accompanied by check circles 1608 that the caregivers may check when they have completed corresponding list item 1606 assigned to the caregivers. Caregiver list page 1700 may be displayed by the application when any of categories of lists 1604 is pressed. Caregiver list page 1700 may include list items 1702 that fit into the chosen category, and list items 1702 may be accompanied by check circles 1704 that the caregivers may check when they (or when they know other caregivers have) completed corresponding list item 1702. List items 1702 may further be accompanied by pictures 1706, and pictures 1706 may show the caregiver assigned to each of list items 1702.

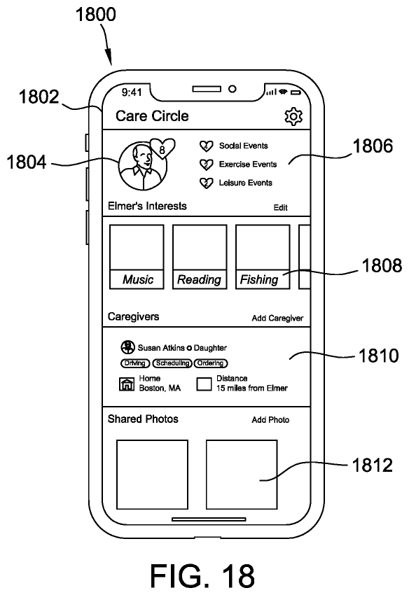

FIG. 18 shows a care circle page 1800 (e.g., which the application may display when button 1222 of FIGS. 12A and 12B is pressed). Care circle page 1800 may include a header 1802, an image 1804, groupings 1806 of events, interests 1808, caregiver profiles 1810, and shared photos 1812. Image 1804 may display an image of the user and/or of any of the caregivers. Groupings 1806 may show an overview of the number and types of events that the user has scheduled in the care calendar. Interests 1808 may allow the caregivers to choose certain interests 1808 of the user such that the application can better prepare a user profile. Caregiver profiles 1810 may show a brief profile of each of the caregivers of the care circle. Shared photos 1812 may show all photos shared by the caregivers of the care circle.

In other embodiments, the application may include additional features and functionality. For example, the application may present a user interface to the user and/or caregivers including an option for the user and/or caregivers to view or input additional data to their profile. The application may additionally provide an option for the user and/or caregivers to input, view, and/or edit medication information for the user. For example, the user and/or caregivers may be able to see the user's daily medication schedule and determine if the user is taking the medication (e.g., through a sensor, as described above).

### Machine Learning & Other Matters

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on mobile computing devices, or associated with smart infrastructure or remote servers), and/or via computer executable instructions stored on non-transitory computer-readable media or medium.

In some embodiments, a care coordination support platform computing device is configured to implement machine learning, such that the care coordination support platform computing device “learns” to analyze, organize, and/or process data without being explicitly programmed. Machine learning may be implemented through machine learning methods and algorithms (“ML methods and algorithms”). In an exemplary embodiment, a machine learning module (“ML module”) is configured to implement ML methods and algorithms. In some embodiments, ML methods and algorithms are applied to data inputs and generate machine learning outputs (“ML outputs”). Data inputs may include but are not limited to: user data, caregiver data, sensor data, assignment data, calendar data, task data, and/or alert data. ML outputs may include but are not limited to: user data, caregiver data, calendar data, task data, and/or assignment data. In some embodiments, data inputs may include certain ML outputs.

In some embodiments, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, combined learning, reinforced learning, dimensionality reduction, and support vector machines. In various embodiments, the implemented ML methods and algorithms are directed toward at least one of a plurality of categorizations of machine learning, such as supervised learning, unsupervised learning, and reinforcement learning.

In one embodiment, the ML module employs supervised learning, which involves identifying patterns in existing data to make predictions about subsequently received data. Specifically, the ML module is “trained” using training data, which includes example inputs and associated example outputs. Based upon the training data, the ML module may generate a predictive function which maps outputs to inputs and may utilize the predictive function to generate ML outputs based upon data inputs. The example inputs and example outputs of the training data may include any of the data inputs or ML outputs described above. For example, a ML module may receive training data comprising user data, caregiver data, and assignment data associated with the user data and caregiver data. The ML module may then generate a model which maps assignment data to aspects of user data and caregiver data. The ML module may then generate assignment data as a ML output based upon subsequently received user data and caregiver data.

In another embodiment, a ML module may employ unsupervised learning, which involves finding meaningful relationships in unorganized data. Unlike supervised learning, unsupervised learning does not involve user-initiated training based upon example inputs with associated outputs. Rather, in unsupervised learning, the ML module may organize unlabeled data according to a relationship determined by at least one ML method/algorithm employed by the ML module. Unorganized data may include any combination of data inputs and/or ML outputs as described above. For example, a ML module may receive unlabeled data comprising user data, caregiver data, and calendar data. The ML module may employ an unsupervised learning method such as “clustering” to identify patterns and organize the unlabeled data into meaningful groups. The newly organized data may be used, for example, to generate a model which associates user data and caregiver data to calendar data.

In yet another embodiment, a ML module may employ reinforcement learning, which involves optimizing outputs based upon feedback from a reward signal. Specifically, the ML module may receive a user-defined reward signal definition, receive a data input, utilize a decision-making model to generate a ML output based upon the data input, receive a reward signal based upon the reward signal definition and the ML output, and alter the decision-making model so as to receive a stronger reward signal for subsequently generated ML outputs. Other types of machine learning may also be employed, including deep or combined learning techniques.

The reward signal definition may be based upon any of the data inputs or ML outputs described above. For example, a ML module may implement reinforcement learning in generating assignment data for caregivers. The ML module may utilize a decision-making model to generate assignment data for caregivers based upon task data, and may further receive user-satisfaction data indicating a level of satisfaction experienced by a user and a caregiver who engaged in a transaction (e.g., the caregiver carrying out a task for the user). A reward signal may be generated by comparing the user-satisfaction data to an assignment score between the user and the caregiver.

Based upon the reward signal, the ML module may update the decision-making model such that subsequently generated assignment scores more accurately predict user satisfaction. For example, the ML module may determine that a specific caregiver has taken the user to four doctor's appointments. The user may enjoy the caregiver taking the user to the doctor's appointments, and the caregiver may enjoy taking the user to the doctor's appointments because the doctor's appointments may be close to the caregiver's house. Therefore, the user and the caregiver may both rate the “transaction” highly. Accordingly, the ML module may learn to automatically assign doctor's appointments to the specific caregiver.

### Exemplary Virtual Care Circle Functionality

In one aspect, a digital solution is provided that will allow seniors stay independent longer in their homes, and that will use technology to create a connected care environment and platform. With the present embodiments, a new digital platform will provide a senior's care circle an easy way to stay connected and help coordinate care virtually. The digital platform may include a new application and chatbot for seniors, and new mobile applications for caregivers/family members/friends to electronically communication, and may be part of a subscription service.

The new, innovative digital platform and application/chatbot may improve the quality of life and care for seniors, and help give their family members added peace of mind, as well as provide unique voice solutions that help seniors' ability to communicate with their care circle from their homes.

In one aspect, the senior will have a voice and touch interface powered by the digital platform and/or caregiver circle application that can help them feel more connected and supported by their care circle, while delivering a personalized experience to them. The digital platform may connect to the care circle mobile app running on mobile device of the care circle members that provides updates and information throughout the day. The care circle can share information back to the senior—creating a virtual circle of support and two-way communication at any time of the day. In one embodiment, the digital platform may include or utilize Amazon's Echo Show™, or similar technologies.

Some of the features utilized by this digital solution include: (1) quick check-in to reassure care circle members; (2) interactive dashboard with scrolling list of daily activities; (3) tools to coordinate key tasks across the care circle; (4) smart-suggestions for events, content, and activities; (5) music and photos sent or delivered to the senior's computing device and/or application; (6) the ability to view the senior's full calendar with audio commands and visual display on their application; (7) social features to help everyone stay connected and up-to-date; and/or other features mentioned elsewhere herein.

The solutions discussed herein will be useful in helping manage care of a loved one, and help family members coordinate care with their aging loved one using a caregiver circle application. The application, chatbot, mobile applications, and digital platform will assist with activities of daily living, transportation, communication, and social connectivity that will be key in helping seniors' ability to “age in place.”

A. Launching the Service

The care circle application, such as for use on mobile devices of caregivers and family members, may facilitate onboarding, setup, and/or profile creation. A family member or a senior may order a product, such as the Echo Show™ product, and download a mobile application to their mobile device. The family member may create a caregiver profile for themselves using the mobile application. Their profile may include their name and a profile photo, and other information and preferences.

The family member may create, using the mobile application, an account for the senior, such as their father or mother, that connects with the family member's profile and/or account. The family member may provide details about, and/or preferences of, the senior using the mobile application, such as the senior's name and a profile photo for the senior. The senior's profile may include other information, such as senior preferences for activities, events, movies, content, tv shows, music, restaurants, service providers, grocery stores, means of transportation, etc.

A sibling of the family member, for example, may also want to be part of the senior's care circle, and the family member may select an “Add Member” icon of the mobile application to add their sibling to the senior's care circle. The family member may also add, via the mobile application, details about their sibling, such as their name and email address, and other information. The family member may add additional caregivers/family members/friends to the senior's care circle using the mobile application.

The mobile application and platform may then send electronic invitations to the mobile devices or email addresses of the members of the care circle added by the originating family member. The electronic invitations may include a link to a mobile application for download that will allow the members of the care circle and/or senior to electronically communicate via the mobile application and digital platform.

The senior may launch or open the application on their computing device and/or on an Alexa-based or other chatbot-based product. After which, the senior may be greeted with a personalized dashboard on their mobile device, tablet, or laptop (or other computing device). The dashboard may include photos of family members, an icon or access to digital photos, an icon or access to a digital or virtual calendar or schedule of events, a “Check In” icon, today's date, and today's scheduled events (such as morning routine, or doctor's appointment). Once the senior opens or launches the application, all connected caregivers or designated caregivers that are part of the senior's care circle may receive an electronic notification that the senior opened the application via the mobile application running on their mobile devices. After which, as the senior provides updates (e.g., went to doctor, need groceries, etc.) on his/her activities/events and well-being via the application on their device and/or via the chatbot, all caregivers/family members in the senior's care circle may view the senior's updates on their respective mobile applications and mobile devices.

B. Managing the Virtual Care Circle

The care circle application may facilitate virtual care circle management and user invitation. If the senior has friends or neighbors that the senior wants to be able to participate in the senior's care circle, the senior may add the friend or neighbor to their virtual care circle via their application, mobile device or other computing device, and/or via the chatbot. Additionally or alternatively, one or more family members may add the friend or neighbor to the virtual care circle for the senior via their mobile care circle application and/or mobile device.

The senior and/or the one or more family members may add the friend's or neighbor's contact information, such as electronic email or text address, and other details, and then select the type of electronic notifications and communications that should be shared with the friend or neighbor via the senior's application or chatbot, or the mobile application running on a family member's mobile device, respectively. For instance, the senior and/or family members may want the friend or neighbor to know when help is needed for driving, scheduling, or ordering things, such as groceries or other items, for the senior.

After accepting an electronic invitation to join the senior's care circle, the friend or neighbor may download the mobile application onto their mobile device. The friend or neighbor may then be able to navigate a care circle feed that may consist of digital posts from all of the members in the care circle, as well as from the senior.

For instance, from the care circle feed, the friend or neighbor may be able to see on the mobile application that the original family member has assigned various items or tasks (such as items or task from a virtual to-do list) to the friend or neighbor (e.g., pick up groceries). After those items or tasks have been completed, the friend or neighbor may virtually check them off via the mobile application so that the family member and/or other members of the virtual care circle see that those items have been completed via the mobile application running on their mobile devices. The friend or neighbor may also virtually post an update using the mobile application to make sure everyone in the virtual care circle notices that he or she has completed the items assigned via the mobile application running on their respective mobile devices.

The friend and neighbor may also virtually post text updates about the well-being or health of the senior, and/or other events, via the mobile application. The family member or other members may comment or otherwise respond to the updates via the mobile application and/or their mobile devices.

C. Starting the Day Off Right

The virtual caregiver circle application may facilitate both “proactive” check-ins and “reactive” check-ins. For proactive check-ins, the senior may open the senior living application on their device or using Alexa (or other chatbot), prompting an automatic check-in. Alexa or another chatbot may greet the senior and ask how they are feeling, such as “Good Morning, Elmer. How are you doing this morning?” After which, the senior may respond verbally, and their verbal response may be converted to a text response or message by the application and/or chatbot—such as “I feel fine, no stiffness in my knees.”

After which, the senior may be prompted by the chatbot and/or application to share his check-in with all caregivers, who can then view the senior's virtual post on the care circle feed. For instance, Alexa or another chatbot may ask “Would you like to share this check-in with your care circle?” If the senior decides to share his check-in with all caregivers/family members/friends, or one or more specific individuals, the chatbot or application may post the senior's update to the care circle feed, and then the chatbot may verbally respond to the senior: “Okay, your message has been shared.”

The family members and other members of the care circle may then receive an electronic notification via the mobile application on their mobile devices that the senior has checked-in for the day. After the senior checks-in, the senior may then view their digital dashboard on their computing device, and/or ask the chatbot what activities/events have scheduled for the day. The dashboard may then display a visual of the senior's scheduled activities for the day, and/or the chatbot may verbally detail the activities for the senior, such as “Call Addison at 10:30 am,” or detail the activity by type, time, and location (“Doctor's appointment, St. Joseph Hospital, at 1 pm”).

For reactive check-ins, such as when the senior fails to actively check-in with the application and/or chatbot on their own, one or more family members or other members of the care circle may receive an electronic notification that the senior has not checked-in this morning nor interacted with the application and/or chatbot. After which, a family member or member of the virtual care circle may decide to give the senior a video or telephone call, using the mobile application. The senior may respond to video or telephone call using the application on their computing device or chatbot. For instance, the chatbot may ask the senior if the senior is alright, and the senior may respond—either by conversing with the chatbot or by using their application on their computing device—that the senior was having coffee with a friend this morning, thus providing peace of mind to the family member that the senior is doing fine.

D. Everyone Knows What's Going On

The caregiver circle application may facilitate collaborative scheduling and calendars. For instance, one or more family members/care circle members may be sent reminders about various activities or events of the senior, depending on settings. For instance, a primary family member in the virtual care circle may be sent reminders about doctor appointments for the senior in the morning of the appointment, and a notification of which member in care circle is responsible for ensuring the senior has transportation to the appointment. After the appointment, the member of the care circle responsible for the appointment may virtually post a message providing an update on how the appointment went to the care circle feed and provide digital access to the message to one or more members of the virtual care circle.

For instance, a daughter may virtually post “Dad's post-surgery results look great, range of motion is better than expected” via the mobile application on her mobile device. Other members of the virtual care circle may receive electronic notification of the daughter's update via wireless communication or data transmission and via the mobile application running on their respective mobile devices. For example, the daughter's brother may receive, via his mobile care circle application, an electronic notification that his sister virtually posted an electronic update on the status of their father, such as “Susan posted an update about Dad's Doctor's appointment this morning.” After which, the brother and other members of the virtual care circle may view the daughter's update on the status of the senior (“Dad”) via the mobile application running on their respective mobile devices.

Then, continuing with this example, the daughter may create a virtual follow-up event, such as a follow-up doctor's appointment as a follow-up event, via her mobile application to add to the senior's virtual calendar viewable by one or more of the members of the virtual care circle. After which, one or more members in the virtual care circle may receive, depending upon individual permissions, an electronic notification of the seniors' next medical appointment (e.g., “Susan created a Follow-up Doctor's Appointment event”), and/or view the senior's updated virtual calendar that includes the next medical appointment.

Continuing with this example, the daughter may then designate whom receives electronic notifications of the next medical appointment; assign responsibility for the next medical appointment to one or more members of the virtual care circle; and/or schedule transportation for the senior to the next medical appointment via the mobile application running on her mobile device. After which, the senior may review and/or approve of the scheduled medical appointment, proposed responsible care giver, and/or proposed mode of transportation via their chatbot and/or application.

Additionally or alternatively, after the daughter posts the follow-up doctor's appointment event via her mobile application, a sibling may post a virtual update regarding the event using his mobile application and mobile device. For instance, brother Jake may virtually post “Going to drive Dad to his next appointment. Also, going to send him flowers!” via his mobile application. After Jake purchases the flowers through an online service or provider, such as Amazon, Jake may update the care circle feed and create a virtual event, via his mobile application, to alert the senior, his father in this example, of the delivery time, such as via the senior's chatbot and/or application.

Additionally or alternatively in this example, the service provider remote server may also have certain access to the mobile applications and/or the senior's chatbot and/or application. For instance, the service provider may provide a verbal or audible notice to the senior of a type of delivery and time of delivery via the senior's chatbot and/or application. Further, the mobile applications may provide real-time or near real-time video or images of the products being delivered to the senior—in this example, video or images of flowers being delivered to the senior.

E. Getting Things Done

The caregiver circle application may facilitate collaborative lists. For example, the senior may notice that his/her lawn needs to be mowed. To his/her virtual to-do list, the senior may add “Order landscaping/lawnmowing” via the seniors' chatbot and/or application. For instance, the senior may say “Alexa, open State Farm,” and then “Add mow the lawn to my to-do list.”

The senior's chatbot and/or application may then add mowing the lawn to the senior's virtual to-do list. For example, the senior's chatbot may respond: “Done. ‘Mow the lawn’ has been added to your to-do list.”

After which, family members and/or care circle members may receive an electronic notification that the senior has updated their virtual to-do list. For instance, virtual care circle members may receive, via their respective mobile applications, an electronic message that indicates that the senior has updated their virtual to-do list—such as electronic notification saying “Elmer added an item to his to-do list. Let's help him complete some tasks.”

After which, one or more designated family members and/or care circle members may take or assign responsibility for the item via a “To-do” icon on their respective mobile application, and/or the senior may also assign responsibility for the task via the senior's chatbot and/or application. For example, a primary family member responsible for assigning tasks to various members of the virtual care circle may assign the task to herself/himself, or the senior may assign the task to one of the care circle members via the senior's chatbot.

Then the senior, and/or assigning family member and/or care circle member, may view the listed items and also view who has been assigned and/or accepted responsibility for each virtual to-do item, such as via the senior's chatbot and/or application and/or via the virtual care circle members' mobile application, respectively. As examples, the virtual to-do items may include “Mow the lawn”; “Walk the dog”; “Get mail”; “Schedule an appointment”; “Schedule a gutter cleaning appointment”; “Coordinate a ride's to Dad's doctor's appointment”; “Pick up Dad's medicine at pharmacy”; “Help Dad prepare and file his taxes”; “Find cleaning service for Dad's house”; and/or other to-do items presented via a display or via the voice of a chatbot. The virtual to-do items may include other items, including those mentioned elsewhere herein.

A family member/care circle member may review the senior's virtual calendar via their mobile application, or a machine learning module, model, algorithm, or program may be programmed, to find a time to schedule a virtual to-do item for the senior. Additionally or alternatively, the senior may assign a virtual to-do item to one or more family members/care circle members via the senior's chatbot and/or application. For instance, a primary family member that has access to the senior's virtual calendar may decide upon a lawn service provider, and schedule a time and date to mow the senior's lawn using the mobile application running on their mobile device. The lawn service provider may be selected via the internet, such as selected via Amazon.com. Additionally or alternatively, the lawn service provider's website and/or Amazon.com may also be programmed with functionality to communicate or otherwise interact with the senior via the senior's chatbot to schedule a time for the lawn service provider to mow the senior's lawn.

In at least one embodiment, the calendar events are stored in one or more tables with a plurality of entries and fields. The one or more tables may be stored in the database 118 (shown in FIG. 1). The one or more tables may be stored in multiple databases. Some databases may include duplicate copies of the user's calendar for ease of access. Each entry includes a plurality of fields describing the event. Fields may include, but are not limited to, current event owner, original event owner, created by, created when, date, start time, end time, duration, repeating event, if exported, if imported, calendars with this event, event name, invitees, attendees, location, notes, and or any other information needed. The links between calendars may include a hyperlink or other address where the calendar is located. The links may also include one or more APIs that are provided by a calendar application on the caregiver's device. In some embodiments, the ECSP application 110 tracks which calendars are linked to which events. When there is an update to one of the events, then the ECSP application 110 broadcasts notification messages to all of the linked calendars to have those calendars update that entry. The ECSP application 110 may also receive updates to events from one or more of the calendar applications. When the ECSP application 110 receives an updated event, the ECSP application 110 determines which other calendars are linked and broadcast messages to those calendar applications about the update. The ECSP application 110 also updates the event in the user's calendar. In some embodiments, the ECSP application 110 accesses one or more APIs associated with calendar programs of the caregivers to allow the ECSP application 110 to compare the calendar entries between the caregiver's calendar and the user's calendar to detect differences between events.

Once the to-do item is assigned, electronic reminders may be generated for the senior. For instance, voice-based reminders may be generated via the senior's chatbot. Text or visual-based reminders may be generated and displayed via the senior's application. Voice-based and text or visual-based reminders may also be sent to the mobile applications of one or more family members/care circle members. For instance, on the day of the lawn service, the daughter may be electronically notified via her mobile application when the lawn service will arrive and/or has arrived. Once the to-do items has been completed, the senior's virtual calendar may be updated to such that all virtual care members can see that the items has been completed via their mobile applications.

### Exemplary Virtual Care Circle Platform & Functionality

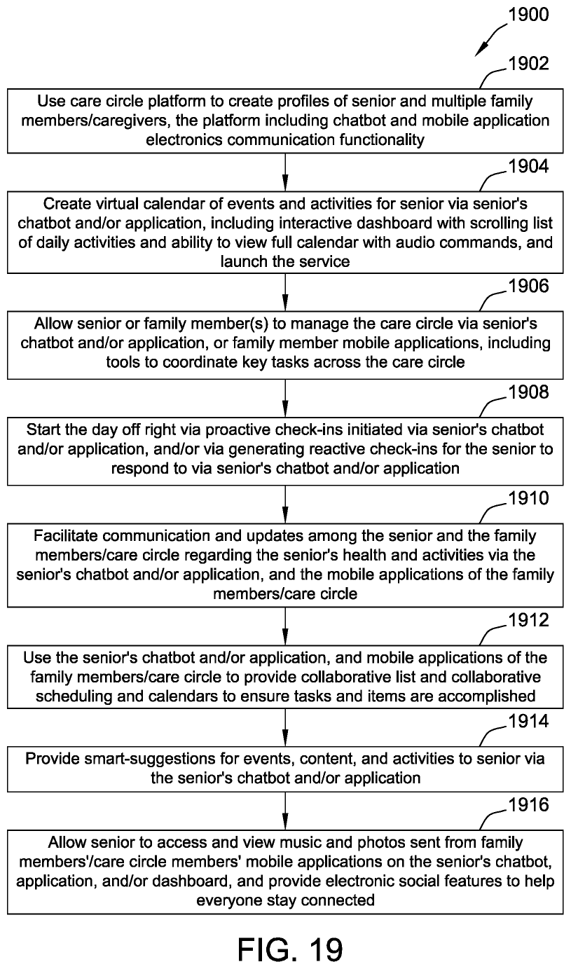

FIG. 19 illustrates an exemplary computer-implemented method 1900 of providing a care circle platform that includes chatbot and mobile application functionality that facilitates coordination of virtual care circle member communication and tasks. The computer-implemented method 1900 may be implemented via one or more processors, transceivers, servers, sensors, applications, mobile applications, chatbots, and related technologies. In some embodiments, method 1900 may be carried out by a digital care circle platform. The digital care circle platform may be substantially similar to, and work in substantially the same way as ECSP server 102 (shown in FIG. 1), described above.

The computer-implemented method 1900 may include, via one or more processors and/or associated transceivers, using 1902 a digital care circle platform to create user profiles and preferences of a senior and multiple family members/caregivers with their permission or affirmative consent. The digital care circle platform may include chatbot functionality, and application and mobile application electronic communication functionality, such as that functionality discussed elsewhere herein, that permits electronic communication via computing devices and/or mobile devices over one or more radio frequency links via wireless communication or data transmission. For instance, the senior may use a chatbot and/or application to enter personal information to create their user profile and/or preferences. Members of the care circle may use a mobile application running on their respective mobile devices to enter personal information to create their respective user profiles.

The computer-implemented method 1900 may include, via one or more processors and/or associated transceivers, creating 1904 a virtual calendar of events and activities for the senior via the chatbot and/or application. The virtual calendar may include an interactive dashboard with a scrolling list of daily activities and events for the senior. The application may include the ability for the senior to view the full calendar after the senior enters one or more audible commands. The method 1900 may include launching the service, which may include activating the chatbot and application of the senior, as well as the mobile applications of the virtual care circle members, once the senior logs into or launches the application for the first time.

The computer-implemented method 1900 may include, via one or more processors and/or associated transceivers, allowing 1906 the senior and/or family members/designated members of the virtual care circle to manage the care circle via the senior's chatbot and/or application, or via the family members'/designated care circle member's mobile application, respectively. The method 1900 may include generating virtual tools that facilitate coordinating key tasks across the virtual care circle, such as assigning specific activities or events to be the responsibility of specific individuals within the virtual care circle.

The computer-implemented method 1900 may include, via one or more processors and/or associated transceivers, initiating 1908 “start the day off right” functionality. For instance, “proactive check-ins” may be generated and/or initiated via the senior interacting with their chatbot and/or application on their computing device. As an example, every morning the senior may check in with their chatbot and/or application at a given time to review their schedule, as well as provide an update to their care circle as to how they are feeling.

The computer-implemented method may also include generating “reactive check-ins” for the senior to respond to via the senior's chatbot and/or application. For instance, if the senior doesn't check in by 8 a.m., the chatbot and/or application may ask the senior if they are doing alright, and the senior may respond to, or converse with, the chatbot verbally or respond via the application textually or by touch.

Additionally or alternatively, a virtual care circle member may send a video, text, or voice message to the senior that the senior receives via their chatbot and/or application. The senior may respond to the virtual care circle member's message via the chatbot and/or application. For instance, a virtual care circle member may send a text message “How are you doing today Dad?” via their mobile application. The senior's application may convert that text message to voice, and the senior's chatbot may verbally ask the senior: “How are you doing today Dad?” At which point, the senior may verbally respond to the chatbot “I am feeling well today. How are you?” After which, the conversation between the senior and the virtual care circle member may continue with the senior interacting with the chatbot to relay messages with the care circle member's mobile application.

The computer-implemented method 1900 may include, via one or more processors and/or associated transceivers, facilitating 1910 electronic and verbal communication and updates among the senior and the family members/care circle members regarding the senior's health/well-being and activities via the senior's chatbot and application, and the mobile application of the respective family members/care circle members. For instance, a virtual “care circle feed” may include updates posted by the senior using their chatbot or the application on their computing device, and/or updates posted by family member's/care circle member's via the mobile application running on their respective mobile devices. The updates may be related to the senior's health, well-being, events, activities, location, etc. The updates may include photos and/or text messages to create timeline of the senior's activities.

The computer-implemented method 1900 may include, via one or more processors and/or associated transceivers, generating, creating, and/or providing 1912 collaborative lists and/or collaborative scheduling to ensure necessary tasks and items are accomplished for the senior. For instance, the method may include allowing the senior to virtually post tasks or events that he/she needs help with completing using their chatbot and/or application. Care circle members may also virtually post tasks that need to be completed via their mobile applications. Care circle members may virtually volunteer for, or accept responsibility for, various tasks via their mobile application. The senior may virtually accept which volunteer care circle member to handle each task, or assign various tasks to specific individuals, via the senior's chatbot and/or application.

The computer-implemented method 1900 may include, via one or more processors and/or associated transceivers, providing 1914 smart-suggestions or recommendations for events, content, and/or activities to the senior via the senior's chatbot and/or application. For instance, based upon “likes” or preferences in the senior's profile, the senior's chatbot and/or application may recommend various events or activities to attend, and/or various online content to view, listen to, or read.

The computer-implemented method 1900 may include, via one or more processors and/or associated transceivers, allowing 1916 the senior to access and view music and photos sent or received from family members'/care circle members' mobile applications via wireless communication or data transmission over one or more radio frequency links. For instance, family members and/or care circle members may push or send photos, music, and/or content to the senior that the senior can review, view, or listen to on the senior's application. The virtual care circle members may also push or send music that the senior can listen to via the senior's chatbot or other digital platform. The computer-implemented method may include additional, less, or alternate functionality, including that discussed elsewhere herein.

### Exemplary Care Coordination Support System

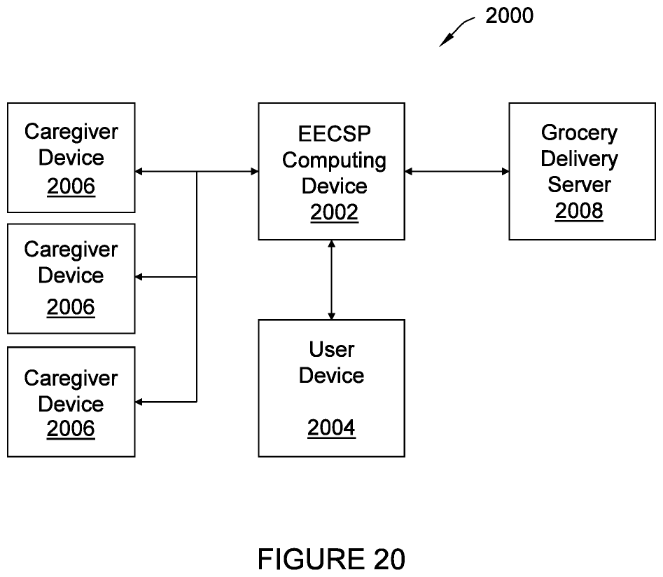

FIG. 20 depicts a view of an exemplary enhanced engagement and care support platform (“EECSP”) system 2000 that may be used to facilitate engagement of a senior user and coordinating care of the senior user between caregivers associated with the senior user that includes an enhancement to allow for grocery delivery for the senior user. In the exemplary embodiment, the EECSP system 2000 may include the ECSP system 100 (shown in FIG. 1) in communication with a grocery delivery server 2008, where the EECSP system 2000 includes a grocery delivery functionality that allows the senior user and/or caregivers in the circle of care of the senior user to order groceries that may be delivered to the senior user. In the exemplary embodiment, the user device 2004 and the caregiver device 2006 may be similar to the client devices 104 (shown in FIG. 1). The EECSP computing device 2002 may be similar to the ECSP computing device 102. And the grocery delivery server 2008 may be similar to the third party server 108 (shown in FIG. 1).

While the terms ‘grocery’ or ‘grocery item’ is used throughout the application, one having skill in the art would understand that these terms can refer to any items to be purchased, such as from a list, including, but not limited to, food, home goods, maintenance supplies, durable goods, healthcare supplies, personal hygiene supplies, first aid supplies, baby care supplies, and/or any other desired or needed item or good.

EECSP system 2000 may include an enhanced engagement and care support platform (“EECSP”) computing device 2002. In the exemplary embodiment, EECSP computing device 2002 may be in communication with a user device 2004 associated with the senior user or individual that is being engaged with, a plurality of caregiver devices 2006 that are each associated with a caregiver in the senior user's circle of care, and a grocery delivery server 2008 capable of taking and processing grocery/shopping orders to be delivered to the senior user. It should be understood that use of the term “grocery delivery service” herein may refer to any grocery or meal delivery service that delivers groceries, meals, and/or home goods to users and/or any combinations thereof.

EECSP system 2000 may facilitate ordering grocery delivery for the senior user (e.g., senior person) by the senior user and/or one or more caregivers in the senior user's circle of care. For example, EECSP system 2000 may pull list items 1606 (shown in FIG. 16) from the lists 1602 (shown in FIG. 16) of the senior user and/or one or more caregivers. EECSP system 2000 may determine which list items 1606 are grocery-related or home good-related items and generate an order for the selected items. EECSP system 2000 may determine for which grocery delivery services the senior user is registered (e.g., by performing a look-up of user data stored in a memory device associated with EECSP system 2000), and EECSP system 2000 may transmit the generated order to one or more grocery delivery servers 2008 such that the order may be fulfilled for the senior user. The grocery delivery servers 2008 may include, for example, servers associated with INSTACART®, SHIPT®, POSTMATES®, UBER EATS®, HELLO FRESH®, BLUE APRON®, local grocery stores, local farmers markets, and any other server associated with any grocery delivery and/or meal delivery services. In the exemplary embodiment, the EECSP computing device 2002 communicates with the grocery delivery server 2008 via one or more APIs (application programming interfaces) provided by the grocery delivery server 2008. The EECSP computing device 2002 may use the APIs to determine current inventory, schedule a delivery and place an order with the grocery delivery server 2008. The EECSP computing device 2002 may have access to a plurality of APIs associated with a plurality of grocery delivery servers 2008.

If EECSP system 2000 determines that the senior user is not signed up for any grocery delivery services, EECSP system 2000 may prompt the senior user (e.g., through user device 2004) or the caregivers (e.g., through caregiver devices 2006) to sign up for one or more grocery delivery services available to the senior user (e.g., that offer services in the area of the senior user). For example, EECSP system 2000 may transmit a notification (e.g., an email, text message, push notification, etc.) to user device 2004 and/or caregiver devices 2006 including a link to one or more grocery delivery services available to the senior user. In some embodiments, EECSP system 2000 may assist the senior user and/or caregivers in registering for the grocery delivery service. In other embodiments, EECSP system 2000 may automatically register the senior user for the grocery delivery service (e.g., using user data including address and payment information stored by EECSP system 2000) when the senior user and/or caregivers give permission for EECSP system 2000 to sign the senior user up for the one or more grocery delivery services.

EECSP system 2000 may arrange for the delivery of groceries based upon user preferences that are input into EECSP system 2000 by the senior user and/or the caregivers. For example, the user preferences may be that the groceries are delivered every week, every two weeks, every time ten or more grocery or home good items are added to the list, etc. In some embodiments, EECSP system 2000 may only order items that are input into EECSP system 2000 (e.g., through the list functionality). In other embodiments, EECSP system 2000 may have a standard order for the senior user and may add any items to the standard order based upon which items the senior user and/or caregivers add to the lists. That is, EECSP system 2000 may have a standard order that is transmitted to grocery delivery server 2008 according to user preferences, and EECSP system 2000 may only edit the list and associated order when the senior user and/or caregivers add items to their lists that are not already included in the standard order.

Once the order has been transmitted to grocery delivery server 2008, EECSP system 2000 may send notification messages to the senior user and/or caregivers regarding the status of the order. In some embodiments, EECSP computing device 2002 may receive messages from grocery delivery server 2008 regarding the order and items included in the order. For example, grocery delivery server 2008 may transmit messages to EECSP computing device 2002 when items of the order must be substituted, or items of the order are no longer available. EECSP system 2000 may transmit the messages from grocery delivery servers 2008 and prompt the senior user and/or caregivers to respond to the messages with a course of action the senior user and/or caregivers would like to take. For example, EECSP system 2000 may transmit a message to the senior user and/or caregivers that only a store brand of an item is available instead of a name brand requested by the senior user and/or caregivers. The message may include a multitude of options available to the senior user and/or caregiver including “Accept substitution,” “Do not accept substitution,” “Order from another service,” and “Order at another time.” Based upon the user input in response to the message, EECSP system 2000 may communicate directly with grocery delivery server 2008 to instruct grocery delivery server 2008 on how to proceed with the order. If the user input requires action from EECSP system 2000 (e.g., if the senior user wishes to order the item from another place or at another time), EECSP system 2000 may store the user input and implement the user request. For example, EECSP system 2000 may submit another order to a different grocery delivery server 2008 and/or flag the item to ensure that the item is added to a subsequent order for the senior user.

In some embodiments, the EECSP system 2000 may convert the grocery/shopping list into a grocery/shopping order. The EECSP system 2000 may convert the grocery/shopping list into a grocery/shopping order in response to a command (either from a senior user or a circle of care member) or on a specific, predetermined day of the week. In some of these embodiments, the senior user may have used one or more generic terms for items on the list, such as, for example, milk, O.J., bread, cheese. For some of these items the EECSP system 2000 may compare the generic item on the list to the items available from each grocery store and present the available choices. Furthermore, the EECSP system 2000 may compare the generic item to the senior user's historical purchases. If the senior user has purchased a quart of 2% milk every week, then the EECSP system 2000 may present that option first. In addition, the EECSP system 2000 may filter the options based on the senior user's medical information or other dietary restrictions. For example, if the senior user is not supposed to have dairy, then the EECSP system 2000 may offer soy and nut-based milk options first to the senior user. In another example, if the senior user added cheese to the grocery/shopping list and the EECSP system 2000 knows that the senior user is vegan, then the EECSP system 2000 may offer vegan cheeses to the senior user. The options and/or choices may be presented to the senior user via a display screen, verbally via the chatbot server 106, and/or both.

In the exemplary embodiment, the EECSP system 2000 uses the grocery/shopping list to generate a grocery/shopping order and marks the ordered items as either ordered or complete. In some embodiments, the EECSP system 2000 waits until the items are delivered until they are marked complete. In some embodiments, the complete items are removed from the senior user's view of the list. Or the items are marked with a strikethrough, check mark, or other acknowledgement that they have been ordered. In some embodiments, the ordered items on the grocery/shopping list are acknowledged with a special marking and remain on the list for a period of time, such as three days. If one or more items may not be ordered, such as from the items being out of stock, those items are left on the list. The EECSP system 2000 may ask the senior user to confirm ordering groceries if the list is short, such as three or fewer items. After generating the grocery/shopping order, the EECSP system 2000 asks the senior user to confirm the grocery/shopping order. When the senior user confirms the order, the EECSP system 2000 places the grocery/shopping order. In a later embodiment, the EECSP system 2000 may store a standard order for the senior user and auto-populate the senior user's grocery/shopping order with the items from the standard order. The standard order may be generated by the EECSP system 2000 based on the historical grocery purchases by the senior user. The EECSP system 2000 may also track the consumption of food by the senior user, and may then remind the senior user to place a grocery/shopping order when food is getting low. The EECSP system 2000 may also determine a day of the week or the month that the senior user normally orders groceries and prompts the senior user asking if they want to order groceries on that day.

In some embodiments, when an item is removed from a list, then the ECSP server 102 and/or the ECSP application 110 alters the items entry in the corresponding table. In some embodiments, the ECSP server 102 and/or the ECSP application 110 adds an entry for the individual that had the item removed from the list. The entry may also include when the item was removed and potentially list a reason that the item was removed. In some embodiments, the entry may indicate that the item was purchased by the caregiver. The item's entry may stay in the corresponding table for a period of time or until acknowledged by the senior user. When displaying the removed item, the senior user interface may display the name of the item with a strikethrough or other mark to indicate that the item has been removed from the corresponding list. The table may also include a field for when the removed item should be completely removed from the list. In other embodiments, the table may include all items placed on the list and when they were removed to allow the senior user and/or one or more caregivers to review previous items.

In some of these embodiments, where the senior user has stated a more specific item, such as cheddar cheese, on the grocery/shopping list, the EECSP system 2000 may use the senior user's historical purchases, dietary restrictions, and/or the available items at the grocery store to add a specific cheddar cheese to the grocery/shopping order. The EECSP system 2000 may determine that the senior user added cheddar cheese to the grocery/shopping list. The EECSP system 2000 may review the senior user's dietary restrictions to note that the senior user is a vegan. The EECSP system 2000 may then review the available items at the grocery store and select a vegan cheddar cheese. If there is more than one vegan cheddar cheese available, the EECSP system 2000 may compare the brands, ingredients, size, and other attributes to make a selection. The EECSP system 2000 may also review the senior user's purchase history to determine one or more of the attributes for making the selection. For example, the senior user's purchase history may show that the senior user is loyal to a specific brand and the EECSP system 2000 may select that brand of vegan cheddar cheese. In another example, the senior user's purchase history may show that the senior user usually buys an 8 oz size of other cheeses, so the EECSP system 2000 may select that size or a size close to that size for the vegan cheddar cheese.

In still further embodiments, the EECSP system 2000 may suggest one or more items for the senior user's grocery/shopping list based on the senior user's purchase history. If the senior user purchased six specific items every week for the last few weeks, the EECSP system 2000 may suggest to the senior user adding those items to the grocery/shopping list. In some embodiments, the EECSP system 2000 may suggest the items through one or more of the chatbot server 106 and/or the touchscreen. If one or more of these items are not on the grocery/shopping list, the EECSP system 2000 may suggest adding the items to the grocery/shopping list or the grocery/shopping order, when the senior user is placing the grocery/shopping order. The EECSP system 2000 may suggest adding items to the grocery/shopping list or grocery/shopping order based on the month, the time of year, the time of month, the day of the week, upcoming holidays, etc.

In some further embodiments, the grocery/shopping order may be placed by one or more caregivers of the circle of care. The caregiver placing the order may add items to the grocery/shopping list before placing the order. The caregiver may order only a portion of the items on the list rather than the entire list. Furthermore, the caregiver may coordinate to pick the grocery/shopping order up from the store. In addition, the caregiver may determine that they over bought some items when they were shopping for themselves and give those items to the senior, and then remove those items from the grocery/shopping list as no longer needed.

In addition, circle of care members could also order delivery food from restaurants for the senior user. For example, a circle of care member may already be ordering pre-made delivery meals for themselves and then also order a meal for the senior user. In some of these embodiments, the circle of care members can see what the senior is planning on having for dinner or other meals. The circle of care members could then order the senior user's desired meal for them or order a different meal for the senior user, such as a special treat. The EECSP system 2000 would then mark that the meal for the senior user has been ordered to prevent multiple meals being ordered for the senior user for that specific meal. In some of these embodiments, the circle of care member may have access to a coupon or discount that allows them to order more than one meal at a discount, so the circle of care member orders one meal for themselves and one for the senior user. In other embodiments, the circle of care member may have a specific meal delivered to the senior user which matches the meal that the circle of care member is having, so that the circle of care member and the senior may share the meal remotely or virtually. This may occur during a holiday, so the family is having the same meal as the senior user as they interact over the devices.

The EECSP system 2000 may also send notifications out to the members of the circle of care when a grocery/shopping order is placed and/or delivered to the senior user. Notifications may also be sent to the senior user when a circle of care member adds an item to the grocery/shopping list. The notification may include an option for the senior to approve putting the item on the grocery/shopping list. Notifications may be sent to members of the senior's circle of care when items are added to the grocery/shopping list, when items are removed from the grocery/shopping list, when a grocery/shopping order is placed, and when the grocery/shopping order is received.

In some embodiments, the senior user may add items to the grocery/shopping list by verbally adding them through the chatbot server 106, by adding them through the app on a smart device, and by scanning an image of the item or a bar code of the item using a smart device. In these embodiments, the senior user can scan an image of the item using a camera in a smart device. The EECSP system 2000 can identify the item based on the packaging of the item. The EECSP system 2000 may perform optical character recognition to determine the name of the item. The EECSP system 2000 may also perform an image search on the image of the packaging to recognize the item. In addition, the EECSP system 2000 may have access to one or more databases to query for one or more UPC or other codes scanned from the packaging.

In some embodiments, the senior user may add items to the grocery/shopping list and/or grocery/shopping order based on a recipe. For example, the senior user may say to the EECSP system 2000 that they want to make spaghetti carbonara for six people on Friday night. The EECSP system 2000 may look up the recipe and determine the ingredients needed. Furthermore, the senior user may store a plurality of recipes in the EECSP system 2000. Or the senior user may request a recipe from the Internet and the EECSP system 2000 accesses the recipe to determine the needed ingredients. In some of these embodiments, the EECSP system 2000 may confirm with the senior user that each ingredient in needed to be ordered. For example, the senior user may have several boxes of spaghetti in their pantry and not need to order spaghetti for the recipe. The EECSP system 2000 may also ensure that the ingredients are ordered in time to allow them to be delivered in time to be used for the planned Friday night meal. In some further embodiments, the EECSP system 2000 may compare the items on the grocery/shopping list to the stored recipes to determine if any items/ingredients are missing. For example, if the senior user has four of the five ingredients for spaghetti carbonara, then the EECSP system 2000 may determine the missing ingredient and ask the senior user if they want to add the missing ingredient to the grocery/shopping list or grocery/shopping order.

In some embodiments, the grocery/shopping list may be associated with a cooking class. In these embodiments, a group of senior users are enrolled in a cooking class. The EECSP system 2000 receives the recipe that will be used for the cooking class and the date of the cooking class. The EECSP system 2000 analyzes the recipe and adds the needed ingredients to the grocery/shopping list. The EECSP system 2000 also sets-up an order for the ingredients to ensure that the ingredients arrive in time for the cooking class. In some embodiments, the senior user sets-up the cooking class with members of their circle of care. This may be grandma showing her grandkids how to cook her famous matzo ball soup. In this embodiment, the members of the circle of care that want to attend the class virtually (through their smart device) and those that wish to attend in person can each register for the class. The EECSP system 2000 may then ensure that there are enough ingredients for all of those that will attend in person. The EECSP system 2000 may also place grocery/shopping orders for the ingredients to be delivered to those that are attending the class virtually. In some further embodiments, those attending the class may be different senior users that wish to cook together, and/or a mix of senior users and circle of care members, potentially from different circles of care.

In some further embodiments, the EECSP system 2000 may automatically reorder grocery items for the senior user. In these embodiments, the senior user may have a standing order of several items that they need every week for their meals, such as, but not limited to, bread, eggs, cheese, milk, fresh fruits, etc. The EECSP system 2000 may then automatically order these items one or more times a week based on the senior user's consumption rate of those items. The consumption rate may be based on the senior user's historical information or the consumption rate may be based on the aggregate information from a plurality of different senior users.

Integration with Multiple Grocery and/or Meal Delivery Services

In some embodiments, EECSP system 2000 may be in communication with multiple grocery delivery servers 2008, and each of the grocery delivery servers 2008 may be associated with a different grocery delivery service. Accordingly, EECSP system 2000 may determine whether the senior user is registered for multiple grocery delivery services and/or may determine whether multiple grocery delivery services are available to the senior user (e.g., based upon comparing the areas served by the grocery delivery services to the location of the senior user). As described above, if it is determined that the senior user is not registered for all grocery delivery services that are available to the senior user, EECSP system 2000 may (i) prompt the senior user and/or caregivers to register for the grocery delivery services and/or (ii) automatically register the senior user for the grocery delivery services with permission from the senior user and/or caregivers. In some embodiments, the registration process for a grocery delivery service takes place outside of the EECSP system 2000. In these embodiments, the senior user may connect an existing registration for a grocery delivery service to their account with the EECSP system 2000.

If the senior user is registered for multiple grocery delivery services, the senior user may input user preferences into EECSP system 2000 related to each grocery delivery service. The user preferences may include, for example, a ranking of the grocery delivery services from most preferred to least preferred, a preference to get certain items only from specific grocery delivery services, and any other preferences associated with the grocery delivery services. For example, if the senior user is registered for grocery delivery services A, B, C, and D, the user preferences may include a ranking of the grocery delivery services from B to C to D to A in order of most preferable to least preferable, a request that home goods are only ordered from A or B, a request that produce is only ordered from B or C, and a request that prescriptions and other health-related items are only ordered from D.

In addition to and/or as an alternative to user preferences, EECSP system 2000 may determine the best grocery delivery services to which the order should be transmitted based upon the availability of the items included in the order for each grocery delivery service. After EECSP system 2000 prepares the order (e.g., by combining the items of the lists) of the senior user, EECSP system 2000 may transmit a query to each grocery delivery server 2008 associated with the grocery delivery services to which the senior user is registered requesting current item inventory listing from each of the grocery delivery services. After the current item inventory from each of the grocery delivery services is received, EECSP system 2000 may compare the items of the order with the received current item inventories. EECSP system 2000 may flag any items that are not in inventory for each of the grocery delivery services, and EECSP system 2000 may automatically transmit the order to the grocery delivery service with the fewest flags. That is, EECSP system 2000 may automatically transmit the order to the grocery delivery server 2008 associated with the grocery delivery service with the most available items on the list.

In some embodiments, if a relatively large number of items are not available at the chosen grocery delivery service, EECSP system 2000 may automatically submit a second order to a different grocery delivery server 2008 associated with a different grocery delivery service such that each item of the order can be fulfilled by the grocery delivery services. Further, if just a few items are not available at the chosen grocery delivery service, EECSP system 2000 may transmit a message to the senior user and/or caregivers requesting user input about the items. For example, the message may include options for EECSP system 2000 to submit an order of the unavailable items to a different grocery delivery service and/or hold off on ordering the items for a subsequent order.

The EECSP system 2000 may place orders at multiple different grocery delivery services to cover all of the items on the list. The EECSP system 2000 may order vegetables from a first grocery delivery service and dry goods from a second grocery delivery service. The EECSP system 2000 may determine how to split up the items on the list between the different grocery delivery services based on user preferences and/or the current inventories for each grocery delivery service. In some embodiments, the EECSP system 2000 may prompt the senior user to agree to ordering from multiple grocery delivery services. In other embodiments, the EECSP system 2000 may automatically place the orders with multiple grocery delivery services.

The EECSP system 2000 may compare the available delivery times to the senior user's calendar to determine when to have the groceries delivered. For example, the EECSP system 2000 may determine not to schedule the grocery delivery on the day that the senior user has a doctor's appointment or is scheduled to be out of the house for most of the day with the garden club. The EECSP system 2000 may determine which grocery delivery service to use based on available delivery times. For example, the EECSP system 2000 may determine to select the grocery delivery service with the soonest available delivery time. The EECSP system 2000 may also determine to select the grocery delivery services with times that are within preference windows in the user preferences. For example, the senior user may request grocery deliveries only between the hours of 1 PM and 5 PM, or any other range or set of ranges of times and/or days of the week.

In some further embodiments, the EECSP system 2000 may be in communication with one or more sensors or Internet of Things (IoT) devices in the senior user's home. In some embodiments, the EECSP system 2000 is in communication with one or more sensors that inform the computer system about the current state of the foods that the senior has. These one or more sensors or Internet of Things (IoT) devices may include but are not limited to, a smart refrigerator, a smart egg tray, smart storage containers, a smart pantry, or any other sensors that assist in gathering grocery information. For example, the sensor may be a smart refrigerator, and can tell the system how many and which foods the senior has in the smart refrigerator. The IoT device may be able to transmit information to the EECSP system 2000 including current quantities of different foods in the refrigerator.

The EECSP system 2000 may then add items to the list based on the current and expected future quantities of food in the refrigerator. For example, the EECSP system 2000 may include historical information about different quantities of different foods in the refrigerator over time to determine one or more trends of consumption of different foods by the senior user. This information may be used to predict when different food items may need to be ordered. The sensors may also include one or more Internet of Things (IoT) devices that monitor food storage, such as, but not limited to an IoT egg tray that may count the number of eggs remaining, IoT food storage containers that may alert users when the contents of their refrigerator are about to spoil, or an IoT pantry that tracks current inventory.

In some embodiments, the one or more sensors may include a scanner or camera that allow the senior user to take a picture of a product or bar code to add that item to the grocery/shopping list. For example, the senior user may scan a bar code or take an image of a type of yogurt that they really enjoyed and want to add to the grocery/shopping list.

In some further embodiments, the senior user may outline or describe their desired different meals for the week to the EECSP system 2000 and the EECSP system 2000 may then determine what is needed to be ordered. For example, the senior user may state that they want pizza on Tuesday night, Tacos on Wednesday night, and spaghetti alla Norma on Thursday night. The EECSP system 2000 may determine the ingredients for each of those meals and place one or more orders for groceries to ensure that the senior user has the proper ingredients for each night. The EECSP system 2000 may also interact with a food delivery service to order one of more of those meals as take-out. For example, the EECSP system 2000 stores a user profile and knows that the senior user prefers to order their pizza from a restaurant rather than make the pizza themselves. In this example, the EECSP system 2000 then orders the pizza to be delivered on Tuesday night.

Integration with Health Data

EECSP system 2000 may store health data of the senior user such that the health data of the senior user may be integrated into the grocery delivery service orders. The health data of the senior user may include, for example, user allergies, user current medications and supplements, user height, weight, and BMI, user health conditions and diseases, and any other health information about the senior user. The health data may be input into EECSP system 2000 by the senior user and/or caregivers.

EECSP system 2000 may compare the health data to items included in the order of the senior user and flag any discrepancies between the items and the health data. For example, the health data of the senior user may include a medication that interacts with grapefruit and may indicate that the senior user has diabetes and is allergic to shellfish. If the items of the order of the senior user include grapefruit, any items with added sugar, or any items including shellfish, EECSP system 2000 may flag these items. In one example, the senior user may have an adverse reaction to products with soy and/or soy lecithin. The EECSP system 2000 scans the ingredients of items to watch out for items containing soy and prioritizing items without soy.

In some embodiments, EECSP system 2000 may transmit a notification to the senior user and/or caregivers prompting user input about the items and noted discrepancies. For example, EECSP system 2000 may prompt the senior user and/or caregivers to determine whether they would like to delete the item from the order and/or give an explanation for the item being included in the order (e.g., if the item is okay to include because the senior user stopped taking a medicine or if the item is okay to include because the senior user is allowed a small amount of added sugar per day). In other embodiments, EECSP system 2000 may automatically delete the items with discrepancies from the order and transmit a notification to the senior user and/or caregivers notifying them of the deletion.

Furthermore, the EECSP system 2000 may adjust suggested items based on the senior user's health information. For example, if the senior user needs to cut back on their sodium, the EECSP system 2000 may suggest low sodium crackers when the senior user requests crackers be ordered from the grocery store.

In some embodiments, the health data may be based on user preferences entered by the senior user. In other embodiments, the health data may be provided by a healthcare provider of the senior user. In still further embodiments, the health data may be provided by one or more members of the senior user's circle of care. To protect the privacy of the health data of the senior user, the EECSP system 2000 may only store the health restrictions and/or preferences of the senior user and not the accompanying medications and/or conditions. For example, the EECSP system 2000 may store that the senior user is on a low sodium diet, but not store that a specific medication that the senior user is taking that is requiring the low sodium diet.

Integration with Models

In some embodiments, the EECSP system 2000 may build one or more models to model the behavior of the senior user. These models may include the consumption rates of various foods and other goods. These models may be based on the historical purchasing information for the senior. Furthermore, the models may be generated using purchasing information from other seniors. The models may then be further enhanced by the purchasing information of the senior in questions. Then these models may be used to determine which goods and services that the senior may need. For example, the EECSP system 2000 may determine that the senior uses one container of shampoo every three weeks and add shampoo to the grocery/shopping list when two weeks are up. In another example, the EECSP computer system 2000 may determine that the senior uses a quart of milk every week and place milk on the grocery/shopping list every week. In some embodiments, the EECSP system 2000 asks the senior to approve putting the item on the grocery/shopping list. In some embodiments, when the senior instructs the EECSP system 2000 to place an order based on the grocery/shopping list, the EECSP system 2000 may analyze the grocery/shopping list and determine one or more items that are needed based on the senior's modeled behavior.

In still further embodiments, the EECSP system 2000 may use machine learning with the modeling to determine when different products need to be reordered. The machine learning could be based on the senior user's historical action, and potentially enhanced with the historical actions of other seniors. The machine learning may also know or learn the expected expiration dates of different foods, both purchased and leftovers. In some embodiments, the EECSP system 2000 may transmit a notification that one or more foods in the refrigerator are no longer safe to eat. In some embodiments, the EECSP system 2000 may determine one or more recommended items that may be presented to the senior user when they are adding items to the grocery/shopping list and/or placing an order. In some further embodiments, the EECSP system 2000 may also present the recommended items to one or more members of the circle of care to have them add those items to the grocery/shopping list. Furthermore, the EECSP system 2000 may learn which stores the senior likes to buy which items from. For example, the senior may like to purchase fresh fruits and vegetables from one store and dry goods from another.

In some further embodiments, the EECSP system 2000 may use a combination of entered information, such as user preferences, and machine learning to generate a virtual profile for the senior user. For example, when the senior user registers with the EECSP system 2000, the EECSP system 2000 asks a plurality of questions to the senior user about their preferences. The EECSP system 2000 may also analyze the interactions with the senior user to determine the senior user's preferences and adds those preferences to the senior user's virtual profile. For example, a senior user may ask the EECSP system 2000 on a daily basis what the weather is like in another city. The EECSP system 2000 may then tell the senior user what the weather is like in that city in the morning after the EECSP system 2000 tells the senior user the weather in their current city. This may be a situation where the senior user spends several months of the year in one city and the other months of the year in the other. Then the EECSP system 2000 may tell the senior user what the weather is like in both their current city and their other city. The EECSP system 2000 may determine that the senior user likes to play soft jazz around 6 PM when they are making dinner and the EECSP system 2000 may update the senior user's virtual profile to start playing soft jazz automatically around 6 PM.

In some further embodiments, the EECSP system 2000 may ask the senior user questions over time, such as during a check-in, to learn the senior user's preferences. The EECSP system 2000 may also ask questions of the circle of care members to learn more about the senior user's preferences. In some embodiments, the initial senior user preferences may be set-up by one or more of the circle of care members.

In additional embodiments, the EECSP system 2000 may store a standard grocery/shopping order for the senior user and associate the standard order with the senior user profile. The standard order may initially be set-up by the senior user. The senior user may then request that the EECSP system 2000 place their standard order or add one or more items to their standard order. The standard order may act as more of a base, or starter, for the grocery/shopping list. However, over time, the EECSP system 2000 may notice one or more changes to the standard order. For example, if the senior user has to lower sodium in their diet, the standard order may be adjusted to account for this change. Furthermore, if a senior user asks to remove an item from a grocery/shopping order more than once, the EECSP system 2000 may remove that item from the standard order. If the senior user added a specific item to the grocery/shopping list more than once, then the EECSP system 2000 may add that specific item to the standard order. In some further embodiments, the initial standard order may be determined by the EECSP system 2000 based on the purchase history of the senior user to determine which items they normally order.

In some embodiments, the EECSP system 2000 may determine what the senior user eats for different meals and detects to see if there are any patterns. If the senior user normally has yogurt and fruit for breakfast, then the EECSP system 2000 ensures that fruit and yogurt are ordered for the senior user. If the EECSP system 2000 determines that the senior user usually makes pancakes on Sundays, then the EECSP system 2000 may place a grocery/shopping order to ensure that the senior user has the needed ingredients by Sunday.

Furthermore, the EECSP system 2000 may determine that the senior user usually orders on a specific day of the week, the EECSP system 2000 may ask on that day if the senior user wants to place an order. In addition, if the weather forecast calls for bad weather on their usual food ordering day, the EECSP system 2000 may ask the senior user if they want to place their normal grocery/shopping order early to avoid the bad weather.

### Exemplary Grocery/Shopping Ordering Process

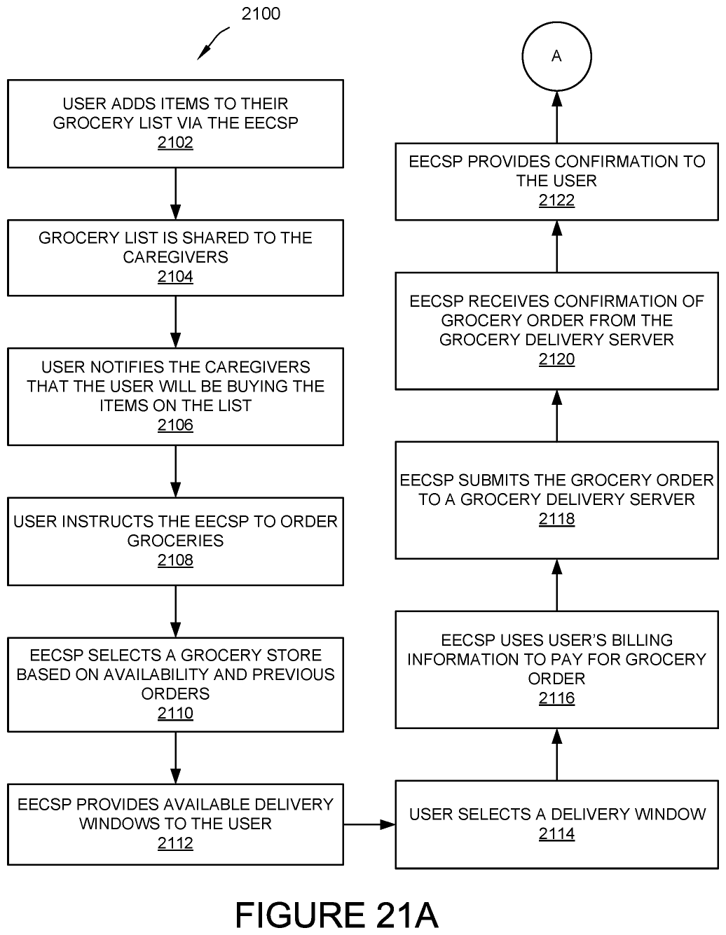

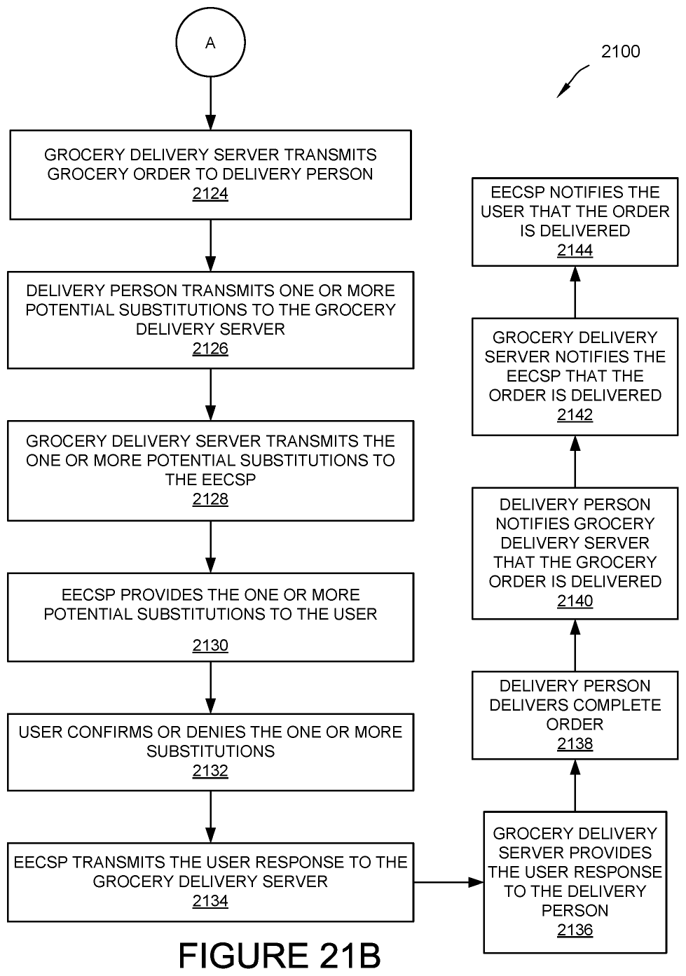

FIGS. 21A and 21B illustrate a flow chart of an exemplary computer-implemented method 2100 for ordering and having groceries delivered implemented by the exemplary enhanced engagement and care support computer system 2000 (shown in FIG. 20). In the exemplary embodiment, method 2100 may be implemented by the EECSP system 2000, with different steps of the method 2100 being implemented by one or more of the user device 2004, the caregiver device 2006, the EECSP computing device 2002, and the grocery delivery server 2008 (all shown in FIG. 20).

In the exemplary embodiment, the senior user adds 2102 items to their grocery/shopping list via their user device 2004 and the EECSP computing device 2002. The senior user may add 2102 the items verbally with the chatbot server 106 or via a graphical user interface on the screen of their user device 2004. The EECSP computer device 2002 may share 2104 the grocery/shopping list with the caregivers via their corresponding caregiver devices 2006. In some other embodiments, one or more of the caregivers may volunteer to purchase one or more items on the grocery/shopping list.

In the exemplary embodiment, the senior user notifies 2106 the caregivers that the senior user will be buying the items on the current grocery/shopping list. The senior user may select an option via their user device 2004 to purchase the item. The EECSP computing device 2002 transmits notifications to the caregiver devices 2006 notifying 2106 the caregivers that the senior user had decided to order the items on their grocery/shopping list.

In the exemplary embodiment, the senior user instructs 2108 the EECSP computing device 2002 to order the groceries. The EECSP computing device 2002 takes the items on the grocery/shopping list to create a grocery/shopping order. The EECSP computing device 2002 communicates with the grocery delivery server 2008 to determine availability of the items on the grocery/shopping list. In some embodiments, the EECSP computing device 2002 may communicate with multiple grocery delivery servers 2008 to determine availability of the items on the grocery/shopping list. Then the EECSP computing device 2002 selects 2110 a grocery store based on the availability of the items and the previous orders made by the senior user. For example, if the senior user normally purchases fresh vegetables from store A, then the EECSP computing device 2002 will select store A if most of the items on the grocery/shopping list are fresh vegetables. If the senior user has stored one or more preferences or used a specific store more than other, then the EECSP computing device 2002 may select 2110 the preferred store. In some other embodiments, the EECSP computing device 2002 may select 2110 a grocery store that has more of the items available or has available delivery windows.

In the exemplary embodiment, the EECSP computing device 2002 communicates with the grocery delivery server 2008 to determine the available delivery windows and then provides 2112 the available delivery windows to the senior user. The senior user selects 2114 a delivery window. The EECSP computing device 2002 uses 2116 the senior user's billing information to pay for the grocery/shopping order. The EECSP computing device 2002 submits 2118 the grocery/shopping order to the grocery delivery server 2008. In some embodiments, the EECSP computing device 2002 may use information from the senior user's registration to submit 2118 the grocery/shopping order. The EECSP computing device 2002 may retrieve the necessary information for the order from the user information, such as address and billing information. The EECSP computing device 2002 may also store preference information such as preferred grocery stores, preferred brands, preferred delivery windows, preferred payment methods, etc. The EECSP computing device 2002 receives 2120 confirmation of the grocery/shopping order from the grocery delivery server 2008. The EECSP computing device 2002 provides 2122 the confirmation to the senior user. In the exemplary embodiment, the senior user has an account with the grocery delivery server 2008 and the EECSP computing device 2002 uses the senior user's account to submit 2118 the grocery/shopping order.

In the exemplary embodiment, the grocery delivery server 2008 transmits 2124 the grocery/shopping order to the computer device of a delivery person. In some situations, the delivery person may determine that one or more items on the grocery/shopping order are unavailable. In these situations, the delivery person may suggest one or more potential substitutions for the unavailable items. The substitutions may include different sized items, different brands, or other potential substitutions to replace the unavailable items. The grocery delivery server 2008 receives 2126 the one or more potential substitutions from the delivery person via their computer device. The grocery delivery server 2008 transmits 2128 the one or more potential substitutions to the EECSP computing device 2002. The EECSP computing device 2002 transmits 2130 the one or more potential substitutions to the user device 2004. The senior user confirms or denies 2132 the one or more substitutions via the user device 2004. The EECSP computing device 2002 transmits 2134 the user response to the grocery delivery server 2008. The grocery delivery server 2008 provides 2136 the user response to the delivery person. In some embodiments, an individual puts together the order and another individual delivers the groceries.

In the exemplary embodiment, when the grocery/shopping order is complete, the delivery person delivers 2138 the complete order to the senior user's residence. The delivery person notifies 2140 the grocery delivery server 2008 that the grocery/shopping order has been delivered. The grocery delivery server 2008 notifies 2142 the EECSP computing device 2002 that the completed grocery/shopping order has been delivered. The EECSP computing device 2002 notifies 2144 the user device 2004 that the completed grocery/shopping order has been delivered.

In some further embodiments, one or more of the caregivers have volunteered to cover all or a portion of the cost of the senior user's grocery/shopping orders. The EECSP computing device 2002 may then use the caregiver's billing information for the grocery/shopping order.

In still further embodiments, the above described method could also be used for pre-made meal delivery services, such as restaurants and other food delivery services.

### Exemplary Virtual Care Circle Embodiments

In one aspect, a digital care circle platform for electronic communication (i) within a virtual care circle, and (ii) between a senior's chatbot and application, and a mobile application running on multiple care circle members' respective mobile devices may be provided. The digital care circle platform may include one or more processors, servers, sensors, wearables, and/or transceivers configured for wireless communication and/or data transmission over one or more radio frequency links between and/or among the senior's chatbot and application, and the mobile application running on each virtual care circle member's mobile device. The digital care circle platform may include, or be interconnected with on communication with, (i) a chatbot associated with the senior configured to receive one or more audible or verbal commands from the senior; (ii) an application associated with a computing device of the senior, the application electronically interacting with and/or communicating with the chatbot; and/or (iii) a mobile application running on each virtual care circle member's mobile device and associated with virtual care circle members, the mobile application configured to electronically communicate with the senior's chatbot and application running on the senior's computing device, such as via wireless communication or data transmission over one or more radio frequency links.

The digital care circle platform may be configured to accept “event” posts from the senior via the chatbot and application, and from each virtual care circle member via the mobile device running on their respective mobile devices. The digital care circle platform may be configured to detect pro-active check-ins that are automatically detected and/or generated by the senior verbally or audibly interacting with the chatbot and/or the senior accessing, viewing, or otherwise interacting with the application running on the senior's computing device. Once a pro-active check-in is detected, the digital care circle platform may be configured to generate an electronic communication detailing the pro-active check-in as a “pro-active check-in event,” and (i) automatically virtually post the pro-active check-in event to a care circle feed access via the mobile application running on one or more virtual care circle member mobile devices, or (ii) otherwise transmit the electronic communication to the mobile application running on one or more virtual care circle member mobile devices to facilitate providing communication on the senior's current activity to the members of the virtual care circle and quick check-in functionality. The digital care circle platform may include additional, less, or alternate functionality, including that discussed elsewhere herein.

For instance, the digital care circle platform may be configured to, via one or more processors, sensors, servers, wearables, and/or transceivers: (a) actively monitor use of, and/or interaction with, the chatbot, application, and/or computing device by the senior; (b) detect that the senior has not used or interacted with the chatbot, application, and/or computing device for a predetermined amount of time; and (c) if so, automatically generate an electronic message to the senior, and transmit or send the electronic message to the chatbot, application, and/or computing device of the senior to facilitate quick check-ins and/or determining whether the senior needs assistance. Additionally or alternatively, the digital care circle platform may be configured to, via one or more processors, sensors, servers, wearables, and/or transceivers: when it is detected that the senior has not used or interacted with the chatbot, application, and/or computing device for a predetermined amount of time, automatically generate an electronic notification detailing such, and transmitting or otherwise sending the electronic notification to one or more virtual care circle member mobile devices to facilitate quick check-ins.

The digital care circle platform may be being configured to, via one or more processors, sensors, servers, wearables, and/or transceivers: (a) actively monitor use of, and/or interaction with, the chatbot, application, and/or computing device by the senior; (b) detect that the senior has not used or interacted with the chatbot, application, and/or computing device for a predetermined amount of time; (c) when it is detected that the senior has not used or interacted with the chatbot, application, and/or computing device for a predetermined amount of time, automatically generate an electronic notification detailing such, and transmitting or otherwise sending the electronic notification to one or more virtual care circle member mobile devices; and/or (d) open, access, or create an audible or verbal communication channel between the senior's chatbot and a virtual care circle member mobile device to facilitate a real-time conversation between the senior and the virtual care circle member, and/or quick check-ins.

The digital care circle platform may be configured to, via one or more processors, sensors, servers, wearables, and/or transceivers: scan the internet for preferred content (music, videos, articles, events, activities, etc.) for the senior based the senior's preferences identified or detailed in a virtual profile associated with the senior; and/or push preferred content, or otherwise providing links thereto, to the senior's chatbot and/or the application running on the senior's computing device.

The digital care circle platform may be configured to, via one or more processors, sensors, servers, wearables, and/or transceivers: (a) generate a virtual calendar of scheduled events and/or activities for the senior, the virtual calendar including a scrolling list of scheduled events and/or activities; and/or (b) display the virtual calendar of scheduled events and/or activities for the senior via the application on a display screen of the senior's computing device, and/or audibly or verbally detail the calendar of scheduled events and/or activities for the senior via the chatbot.

The digital care circle platform may be configured to, via one or more processors, sensors, servers, wearables, and/or transceivers: (i) receive or accept audible or verbal commends from the senior via the chatbot regarding details of an event or activity to add to their virtual calendar; and/or (ii) add the event or activity to the senior's virtual calendar.

The digital care circle platform may be configured to, via one or more processors, sensors, servers, wearables, and/or transceivers: (a) receive or accept audible or verbal commends from the senior via the chatbot regarding details of a task or item to add to their virtual to-do list; (b) add the task or item to the senior's virtual to-do list; and/or (c) generate and post an electronic notification detailing the task or item to the care circle feed accessible via the mobile application running on one or more virtual care circle member mobile devices to facilitate coordinating key tasks among the members of the virtual care circle.

The digital care circle platform may be configured to, via one or more processors, sensors, servers, wearables, and/or transceivers: (a) receive or accept audible or verbal commands from the senior via the chatbot regarding identification of a virtual care circle member to assign the task or item to, or responsibility for; and/or (b) generate and post an electronic notification detailing which virtual care circle member has been assigned the task or item to the care circle feed accessible via the mobile application running on one or more virtual care circle member mobile devices.

The digital care circle platform may be configured to, via one or more processors, sensors, servers, wearables, and/or transceivers: (i) receive or accept user input from a virtual care circle member via the mobile application regarding details of a task or item to add to the senior's virtual to-do list; (ii) add the task or item to the senior's virtual to-do list; and/or (iii) generate and post an electronic notification detailing the task or item to the care circle feed accessible via the mobile application running on one or more virtual care circle member mobile devices to facilitate coordinating key tasks among the members of the virtual care circle.

The digital care circle platform may be configured to, via one or more processors, sensors, servers, wearables, and/or transceivers: (i) receive user input from a virtual care circle member via the mobile application regarding identification of a virtual care circle member to assign the task or item to, or responsibility for; and/or (ii) generate and post an electronic notification detailing which virtual care circle member has been assigned the task or item to the care circle feed accessible via the mobile application running on one or more virtual care circle member mobile devices. The digital care circle platform may also be configured to, via one or more processors, sensors, servers, and/or transceivers: allow virtual care circle members to take or assign responsibility for various tasks or items in a virtual to-do list associated with the senior to facilitate collaborative scheduling and coordinating key tasks across the virtual care circle.

In another aspect, a computer-implemented method for electronic communication (i) within a virtual care circle, and (ii) between a senior's chatbot and application, and a mobile application running on multiple care circle members' respective mobile devices, the method may include, via one or more processors, servers, sensors, wearables, digital platforms, and/or transceivers configured for wireless communication and/or data transmission over one or more radio frequency links between and/or among the senior's chatbot and application, and the mobile application running on each virtual care circle member's mobile device: (1) receiving one or more one or more audible or verbal commands from the senior via the chatbot; (2) electronically interacting with and/or communicating with the chatbot via an application associated with a computing device of the senior; (3) electronically communicating with the senior's chatbot and application running on the senior's computing device via a mobile application running on each virtual care circle member's mobile device and associated with virtual care circle members, such as via wireless communication or data transmission over one or more radio frequency links; (4) electronically accepting or wirelessly receiving event posts from the senior via the chatbot and application, and from each virtual care circle member via the mobile device running on their respective mobile devices, such as via wireless communication or data transmission over one or more radio frequency links; (5) detecting pro-active check-ins that are automatically detected and/or generated by the senior verbally or audibly interacting with the chatbot and/or the senior accessing, viewing, or otherwise interacting with the application running on the senior's computing device; and/or (6) once a pro-active check-in is detected, generating an electronic communication detailing the pro-active check-in as a pro-active check-in event, and (a) automatically virtually posting the pro-active check-in event to a care circle feed access via the mobile application running on one or more virtual care circle member mobile devices, and/or (b) otherwise transmitting the electronic communication to the mobile application running on one or more virtual care circle member mobile devices to facilitate providing communication on the senior's current activity to the members of the virtual care circle and quick check-in functionality. The method may include additional, less, or alternate functionality, including that discussed elsewhere herein.

For instance, the computer-implemented method may include, via one or more processors, sensors, servers, wearables, and/or transceivers: actively monitoring use of, and/or interaction with, the chatbot, application, and/or computing device by the senior; detecting that the senior has not used or interacted with the chatbot, application, and/or computing device for a predetermined amount of time; and if so, automatically generating an electronic message to the senior, and transmitting or sending the electronic message to the chatbot, application, and/or computing device of the senior to facilitate quick check-ins and/or determining whether the senior needs assistance. The method may also include, via one or more processors, sensors, servers, wearables, and/or transceivers: when it is detected that the senior has not used or interacted with the chatbot, application, and/or computing device for a predetermined amount of time (such as 12 hours, 18 hours, 24 hours, 48 hours, etc.), automatically generating an electronic notification detailing such, and transmitting or otherwise sending the electronic notification to one or more virtual care circle member mobile devices to facilitate quick check-ins.

The computer-implemented method may include, via one or more processors, sensors, servers, wearables, and/or transceivers: actively monitoring use of, and/or interaction with, the chatbot, application, and/or computing device by the senior; detecting that the senior has not used or interacted with the chatbot, application, and/or computing device for a predetermined amount of time; when it is detected that the senior has not used or interacted with the chatbot, application, and/or computing device for a predetermined amount of time (e.g., 24 hours, 48 hours, etc.), automatically generating an electronic notification detailing such, and transmitting or otherwise sending the electronic notification to one or more virtual care circle member mobile devices; and/or opening, accessing, or creating an audible or verbal communication channel between the senior's chatbot and a virtual care circle member mobile device to facilitate a real-time conversation between the senior and the virtual care circle member, and/or quick check-ins.

The computer-implemented method may include, via one or more processors, sensors, servers, wearables, and/or transceivers: scanning or searching the internet for preferred content (music, videos, articles, events, activities, etc.) for the senior based the senior's preferences identified or detailed in a virtual profile associated with the senior; and/or pushing preferred content, or otherwise providing links thereto, to the senior's chatbot and/or the application running on the senior's computing device.

The computer-implemented method may include, via one or more processors, sensors, servers, wearables, and/or transceivers: generating a virtual calendar of scheduled events and/or activities for the senior, the virtual calendar including a scrolling list of scheduled events and/or activities; and displaying the virtual calendar of scheduled events and/or activities for the senior via the application on a display screen of the senior's computing device, and/or audibly or verbally detailing the calendar of scheduled events and/or activities for the senior via the chatbot.

The computer-implemented method may include, via one or more processors, sensors, servers, wearables, and/or transceivers: receiving audible or verbal commends from the senior via the chatbot regarding details of an event or activity to add to their virtual calendar; and/or adding the event or activity to the senior's virtual calendar.

The computer-implemented method may include, via one or more processors, sensors, servers, wearables, and/or transceivers: receiving or accepting audible or verbal commends from the senior via the chatbot regarding details of a task or item to add to their virtual to-do list; adding the task or item to the senior's virtual to-do list; and/or generating and posting an electronic notification detailing the task or item to the care circle feed accessible via the mobile application running on one or more virtual care circle member mobile devices to facilitate coordinating key tasks among the members of the virtual care circle. The method may include, via one or more processors, sensors, servers, wearables, and/or transceivers: receiving or accepting audible or verbal commands from the senior via the chatbot regarding identification of a virtual care circle member to assign the task or item to, or responsibility for; and/or generating and posting an electronic notification detailing which virtual care circle member has been assigned the task or item to the care circle feed accessible via the mobile application running on one or more virtual care circle member mobile devices.

The computer-implemented method may include, via one or more processors, sensors, servers, wearables, and/or transceivers: receiving or accepting user input from a virtual care circle member via the mobile application regarding details of a task or item to add to the senior's virtual to-do list; adding the task or item to the senior's virtual to-do list; and/or generating and posting an electronic notification detailing the task or item to the care circle feed accessible via the mobile application running on one or more virtual care circle member mobile devices to facilitate coordinating key tasks among the members of the virtual care circle. The method may include, via one or more processors, sensors, servers, wearables, and/or transceivers: receiving or accepting user input from a virtual care circle member via the mobile application regarding identification of a virtual care circle member to assign the task or item to, or responsibility for; and/or generating and posting an electronic notification detailing which virtual care circle member has been assigned the task or item to the care circle feed accessible via the mobile application running on one or more virtual care circle member mobile devices.

The method may also include, via one or more processors, sensors, servers, wearables, and/or transceivers: allowing virtual care circle members to take or assign responsibility for various tasks or items in a virtual to-do list associated with the senior to facilitate collaborative scheduling and coordinating key tasks across the virtual care circle.

In a further aspect, a user computer device to facilitate senior engagement with their daily schedule and caregivers associated with the seniors, and coordinate care between caregivers, while providing a grocery delivery platform may be provided. The user computer device may include at least one processor and/or transceiver in communication with at least one memory device. The user computer device may be in communication with at least one second computer device. The at least one processor may be programmed to (a) store user registration information for a user associated with the user computing device; (b) store caregiver registration information for a plurality of caregivers associated with the user, wherein the caregiver registration information includes data for identifying the at least one second computer device; (c) receive, from at least one of the user and the plurality of caregivers, one or more items of an order of the user; (d) compile the received items into a list; (e) generate the order based on user preferences and the list; (f) determine at least one third party to fulfill the order based on the items on the list and the user preferences; and/or (g) transmit the order to a third party server associated with the determined third party.

In addition, the order may include at least one of grocery items and home good items. Furthermore, the third party server may be associated with a grocery delivery service that delivers at least one of grocery items and the home good items.

In addition, the at least one processor may be further programmed to determine whether the user is registered for one or more grocery delivery services. When it is determined that the user is registered for one grocery delivery service, the at least one processor may then transmit the list to the third party server associated with the registered grocery delivery service. Furthermore, when it is determined that the user is registered for a plurality of grocery delivery services, the at least one processor may be programmed to (a) determine how many items included on the list that each of the plurality of grocery delivery services can fulfill; (b) select a grocery delivery service of the plurality of grocery delivery services for the order based on a comparison of the determined number of items that can be fulfilled and one or more user preferences; and/or (c) transmit the list to the third party server associated with the selected grocery delivery service.

In addition, the at least one processor may be further programmed to (a) transmit a query to each of third party server associated with the plurality of registered grocery delivery services to request a current item inventory; (b) compare the items included in the current item inventory of each grocery delivery service of the plurality of grocery delivery services with the items included in the order of the user; and/or (c) select one or more grocery delivery services of the plurality of grocery delivery services to fulfill the items included in the order based upon the comparison, where selected one or more grocery delivery services can fulfill the most items included in the order of the user.

Furthermore, when it is determined that the user is not registered for a grocery delivery service, the at least one processor may be further programmed to (a) prompt at least one of the user and the plurality of caregivers to permit registration for the grocery delivery service, and/or (b) automatically populate a registration form for the grocery delivery service based upon the stored user registration information.

In addition, the user registration information includes user preferences including preferred times that the items be fulfilled and preferred third parties with which the items are to be fulfilled.

Furthermore, the at least one processor may be further programmed to (a) receive, from the third party server, that one or more items from the order cannot be fulfilled, and/or (b) transmit a notification to at least one of the user and the plurality of caregivers that the one or more items cannot be fulfilled, where the notification prompts the at least one of the user and at least one caregiver of the plurality of caregivers for user input. In addition, the at least one processor may be further programmed to (a) receive the user input from at least one of the user and the plurality of caregivers, and/or (b) based upon the user input, transmit a list of unfulfilled items to a different third party server to fulfill the list of unfulfilled items.

In addition, the user registration information may include user health information including active prescriptions and allergy information. Furthermore, the at least one processor maybe further programmed to (a) compare the user health information with the items included in the order, and/or (b) determine any discrepancies between the user health information and the items included in the order, where the discrepancies include items included in the order to which the user is allergic and items included in the order that have negative interactions with active prescriptions of the user.

In addition, the at least one processor may be further programmed to notify at least one of the user and the plurality of caregivers of the determined discrepancies and delete items with discrepancies from the order.

In addition, the third party server may be associated with a meal delivery service. The at least one processor may include additional, less, or alternate functionality, including that discussed elsewhere herein.

In an additional aspect, a computer-implemented method for facilitating senior engagement may be provided. The method may be implemented by a computer device including at least one processor and/or transceiver in communication with at least one memory device. The method may include, via the computer device, and/or one or more local or remote processors and associated transceivers, (i) storing user registration information for a user associated with a user computing device; (ii) storing caregiver registration information for a plurality of caregivers associated with the user, wherein the caregiver registration information includes data for identifying at least one second computer device associated with the corresponding caregiver; (iii) receiving, from at least one of the user and the plurality of caregivers, one or more items of an order of the user; (iv) compiling the received items into a list; (v) generating the order based on user preferences and the list; (vi) determining at least one third party to fulfill the order based on the items on the list and the user preferences; and/or (vii) transmitting the order to a third party server associated with the determined third party.

In addition, the order may include at least one of grocery items and home good items. The third party server may be associated with a grocery delivery service that delivers at least one of grocery items and home good items.

Furthermore, the method may include determining whether the user is registered for one or more grocery delivery services. In addition, when it is determined that the user is registered for one grocery delivery service, the method may include transmitting the list to the third party server associated with the registered grocery delivery service.

In addition, when it is determined that the user is registered for a plurality of grocery delivery services, the method may include determining how many items included on the list that each of the plurality of grocery delivery services can fulfill. The method may further include selecting a grocery delivery service of the plurality of grocery delivery services for the order based on a comparison of the determined number of items that can be fulfilled and one or more user preferences. In addition, the method may include transmitting the list to the third party server associated with the selected grocery delivery service.

In addition, the method may include transmitting a query to each of third party server associated with the plurality of registered grocery delivery services to request a current item inventory. The method may further include comparing the items included in the current item inventory of each grocery delivery service of the plurality of grocery delivery services with the items included in the order of the user. In addition, the method may include selecting one or more grocery delivery services of the plurality of grocery delivery services to fulfill the items included in the order based upon the comparison, wherein selected one or more grocery delivery services can fulfill the most items included in the order of the user. The method may include additional, less, or alternate functionality, including that discussed elsewhere herein.

### ADDITIONAL CONSIDERATIONS

With the foregoing, users and caregivers may opt-in or register to a care coordination support platform program or other type of program. After the users and caregivers give their affirmative consent or permission, a care coordination support platform remote server may collect data from the mobile devices, user computing devices, smart home controllers, smart vehicles, autonomous or semi-autonomous vehicles, smart infrastructure, smart buildings, smart aerial devices (e.g., drones), and/or other smart devices, such as with the permission or affirmative consent of the users and caregivers. The data collected may be related to user activities and/or user/caregiver schedules and current locations.

As will be appreciated based upon the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In one embodiment, a computer program is provided, and the program is embodied on a computer readable medium. In an exemplary embodiment, the system is executed on a single computer system, without requiring a connection to a sever computer. In a further embodiment, the system is being run in a Windows® environment (Windows is a registered trademark of Microsoft Corporation, Redmond, Wash.). In yet another embodiment, the system is run on a mainframe environment and a UNIX® server environment (UNIX is a registered trademark of X/Open Company Limited located in Reading, Berkshire, United Kingdom). The application is flexible and designed to run in various different environments without compromising any major functionality. In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes.

In some embodiments, registration of users for the care coordination support platform includes opt-in informed consent of users to data usage by the smart home devices, wearable devices, mobile devices, autonomous vehicles, and/or smart vehicles consistent with consumer protection laws and privacy regulations. In some embodiments, the user data, the caregiver data, and/or other collected data may be anonymized and/or aggregated prior to receipt such that no personally identifiable information (PII) is received. In other embodiments, the system may be configured to receive user and caregiver data and/or other collected data that is not yet anonymized and/or aggregated, and thus may be configured to anonymize and aggregate the data. In such embodiments, any PII received by the system is received and processed in an encrypted format, or is received with the consent of the individual with which the PII is associated. In situations in which the systems discussed herein collect personal information about individuals, or may make use of such personal information, the individuals may be provided with an opportunity to control whether such information is collected or to control whether and/or how such information is used. In addition, certain data may be processed in one or more ways before it is stored or used, so that personally identifiable information is removed.

As used herein, an element or step recited in the singular and proceeded with the word “a” or “an” should be understood as not excluding plural elements or steps, unless such exclusion is explicitly recited. Furthermore, references to “exemplary embodiment” or “one embodiment” of the present disclosure are not intended to be interpreted as excluding the existence of additional embodiments that also incorporate the recited features.

The patent claims at the end of this document are not intended to be construed under 35 U.S.C. § 112(f) unless traditional means-plus-function language is expressly recited, such as “means for” or “step for” language being expressly recited in the claim(s).

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An engagement and care support platform (“ECSP”) computer device comprising at least one processor in communication with at least one memory device, the ECSP computer device in communication with a first client device and a plurality of second client devices, the at least one processor of the ECSP computer device is programmed to:
store user registration information for a senior user associated with the first client device;
store caregiver registration information for a plurality of caregivers associated with the senior user, wherein the caregiver registration information includes data for identifying the plurality of second client devices;
receive, from the senior user and at least one of the plurality of caregivers, a plurality of items of an order of the senior user;
compile the received items into a list;
generate the order based on user preferences and the list;
determine at least one third party to fulfill the order based on the items on the list and the user preferences; and
transmit the order to a third-party server associated with the determined third party.

2. The ECSP computer device of claim 1, wherein the order includes at least one of grocery items and home good items, and wherein the third-party server is associated with a grocery delivery service that delivers at least one of grocery items and the home good items.

3. The ECSP computer device of claim 2, wherein the at least one processor is further programmed to determine whether the senior user is registered for one or more grocery delivery services.

4. The ECSP computer device of claim 3, wherein the at least one processor is further programmed to when it is determined that the senior user is registered for one grocery delivery service, transmit the list to the third-party server associated with the registered grocery delivery service.

5. The ECSP computer device of claim 3, wherein the at least one processor is further programmed to:
when it is determined that the senior user is registered for a plurality of grocery delivery services, determine how many items included on the list that each of the plurality of grocery delivery services can fulfill;
select a grocery delivery service of the plurality of grocery delivery services for the order based on a comparison of the determined number of items that can be fulfilled and one or more user preferences; and
transmit the list to the third-party server associated with the selected grocery delivery service.

6. The ECSP computer device of claim 5, wherein the at least one processor is further programmed to:
transmit a query to each of third-party server associated with the plurality of registered grocery delivery services to request current inventory information;
compare the items included in the current inventory information of each grocery delivery service of the plurality of grocery delivery services with the items included in the order of the senior user; and
select one or more grocery delivery services of the plurality of grocery delivery services to fulfill the items included in the order based upon the comparison, wherein selected one or more grocery delivery services can fulfill the most items included in the order of the senior user.

7. The ECSP computer device of claim 6, wherein the at least one processor is further programmed to:
generate a first order for a first grocery delivery service to include available items based on the current inventory information of the first grocery delivery service; and
generate a second order for a second grocery delivery service to include available items based on the current inventory information of the second grocery delivery service.

8. The ECSP computer device of claim 3, wherein the at least one processor is further programmed to:
when it is determined that the senior user is not registered for a grocery delivery service, prompt at least one of the senior user and the plurality of caregivers to permit registration for the grocery delivery service; and
automatically populate a registration form for the grocery delivery service based upon the stored user registration information.

9. The ECSP computer device of claim 1, wherein the user registration information includes user preferences including preferred times that the items be fulfilled and preferred third parties with which the items are to be fulfilled.

10. The ECSP computer device of claim 1, wherein the at least one processor is further programmed to:
receive, from the third-party server, that one or more items from the order cannot be fulfilled; and
transmit a notification to at least one of the senior user and the plurality of caregivers that the one or more items cannot be fulfilled, wherein the notification prompts the at least one of the senior user and at least one caregiver of the plurality of caregivers for user input.

11. The ECSP computer device of claim 10, wherein the at least one processor is further programmed to:
receive the user input from at least one of the senior user and the plurality of caregivers; and
based upon the user input, transmit a list of unfulfilled items to a different third-party server to fulfill the list of unfulfilled items.

12. The ECSP computer device of claim 1, wherein the user registration information includes user health information including active prescriptions and allergy information.

13. The ECSP computer device of claim 12, wherein the at least one processor is further programmed to:
compare the user health information with the items included in the order; and
determine any discrepancies between the user health information and the items included in the order, wherein the discrepancies include items included in the order to which the senior user is allergic and items included in the order that have negative interactions with active prescriptions of the senior user.

14. The ECSP computer device of claim 13, wherein the at least one processor is further programmed to notify at least one of the senior user and the plurality of caregivers of the determined discrepancies.

15. The ECSP computer device of claim 14, wherein the at least one processor is further programmed to delete items with discrepancies from the order.

16. The ECSP computer device of claim 1, wherein the third-party server is associated with a meal delivery service.

17. The ECSP computer device of claim 1, wherein the at least one processor is further programmed to:
determine one or more items on the list that could include a plurality of options;
determine one or more suggestions for the one or more items based on the user preferences and availability of items at the at least one third party; and
present the one or more suggestions to the senior user.

18. The ECSP computer device of claim 1, wherein the at least one processor is further programmed to:
store a plurality of items in a standard order;
create a new grocery list by adding the plurality of items in the standard order; and
present the new grocery list to the senior user.

19. The ECSP computer device of claim 18, wherein the at least one processor is further programmed to:
compare a plurality of historical grocery orders associated with the senior user;
determine a plurality of commonly ordered items based on the comparison; and
generate the standard order based on the plurality of commonly ordered items.

20. The ECSP computer device of claim 18, wherein the at least one processor is further programmed to:
determine a current amount of food in the senior user's home;
determine if the current amount of food in the senior user's home is below a predetermined threshold; and
if the current amount of food in the senior user's home is below a predetermined threshold, place a grocery order for the plurality of items in the standard order.

21. The ECSP computer device of claim 20, wherein the at least one processor is further programmed to:
determine a food consumption rate for the senior user based on the plurality of historical grocery orders; and
determine the current amount of food in the senior user's home based on the determined food consumption rate.

22. The ECSP computer device of claim 21, wherein the at least one processor is further programmed to:
receive food information from a plurality of sensors; and
determine the current amount of food in the senior user's home based on the food information from the plurality of sensors.

23. The ECSP computer device of claim 20, wherein if the current amount of food in the senior user's home is below the predetermined threshold, the at least one processor is further programmed to ask the senior user if they want to place a grocery order.

24. The ECSP computer device of claim 1, wherein the at least one processor is further programmed to:
determine a day that the senior user usually orders groceries based on a plurality of historical user grocery orders; and
ask the senior user if they want to place a grocery order on the determined day.

25. A computer-implemented method for facilitating senior engagement, the method is implemented by a computer device comprising at least one processor in communication with at least one memory device, the computer device in communication with a first client device and a plurality of second client devices, the method comprises:
storing user registration information for a senior user associated with a first client device;
storing caregiver registration information for a plurality of caregivers associated with the senior user, wherein the caregiver registration information includes data for identifying the plurality of second client devices associated with the corresponding caregiver;
receiving, from the senior user and at least one of the plurality of caregivers, a plurality of items for an order of the senior user;
compiling the received items into a list;
requesting current inventory information from a plurality of item delivery services;
generating a first order for a first item delivery service of the plurality of item delivery services to include available items based on the current inventory information of the first grocery delivery service, user preferences, and the list;
transmitting the first order to the first item delivery service;
generating a second order for a second item delivery service of the plurality of item delivery services to include available items based on the current inventory information of the second item delivery service, user preferences, and remaining items on the list; and
transmitting the second order to the second item delivery service.

26. The computer-implemented method of claim 25, wherein the first order includes at least one of grocery items and home good items, and wherein the first item delivery service is associated with a grocery delivery service that delivers at least one of grocery items and home good items.

27. The computer-implemented method of claim 26 further comprising determining whether the senior user is registered for one or more item delivery services of the plurality of item delivery services.

28. The computer-implemented method of claim 27 further comprising when it is determined that the senior user is registered for one item delivery service, transmitting the list to a third party server associated with the registered item delivery service.
